(19) United States
(12) Patent Application Publication
(10) Pub. No.: US 2026/0216518 A1
(43) Pub. Date: Jul. 30, 2026

(54) PROGRAMMING SACRAL STIMULATION USING EVOKED SIGNALS

(71) Applicant: Medtronic, Inc., Minneapolis, MN (US)

(72) Inventors: Hank T. Bink, Golden Valley, MN (US); Julia P. Slopsema, Minneapolis, MN (US); Lisa M. Jungbauer Nikolas, Lino Lakes, MN (US); Nicholas D. Buse, New Brighton, MN (US); Sarah J. Offutt, Golden Valley, MN (US); Simeng Zhang, Golden Valley, MN (US); Joshua O. Usoro, St. Paul, MN (US)

(21) Appl. No.: 19/456,085

(22) Filed: Jan. 22, 2026

Related U.S. Application Data
(60) Provisional application No. 63/749,396, filed on Jan. 24, 2025.

(51) Int. Cl. A61N 1/36; A61N 1/05

(52) U.S. Cl. CPC A61N 1/36139; A61N 1/0551; A61N 1/36125; A61N 1/36185

(57) ABSTRACT

An example system includes processing circuitry configured to: control stimulation circuitry to deliver an electrical stimulus to a patient; control sensing circuitry to sense an electrical electrophysiological signal after the electrical stimulus is delivered; compare a first portion of the electrical signal within a first sensing window to a second portion of the electrical signal within a second sensing window, wherein the first sensing window occurs prior to the second sensing window; identify, based on the comparison of the first portion to the second portion, an evoked response within the first sensing window that was elicited by the electrical stimulus; and determine, based on the evoked response identified from the electrical signal, a set of electrodes of a plurality of electrodes as stimulation electrodes.

This application claims the benefit of, and priority to, U.S. Provisional Patent Application No. 63/749,396, filed Jan. 24, 2025, the entire contents of which is incorporated herein by reference.

### TECHNICAL FIELD

This disclosure generally relates to electrical stimulation, and more specifically, to programming electrical stimulation based on sensed signals.

### BACKGROUND

Medical devices may be external or implanted, and may sense electrical signals (e.g., neuromuscular signals from central and/or peripheral nerves/muscles) and/or deliver electrical stimulation therapy to various tissue sites of a patient to treat a variety of symptoms or conditions such as, for example, one or more of bladder dysfunction (e.g., retention, overactive bladder, urgency, urgency frequency, urinary incontinence, bladder incontinence, stress incontinence, nocturia, or any other dysfunction of the bladder), bowel dysfunction (e.g., fecal incontinence, intractable constipation, irritable bowel syndrome, inflammatory bowel disease, or any other dysfunction of the bowels), chronic pain, stroke, spinal cord injury, neuropathy, tremor, Parkinson's disease, multiple sclerosis, other movement disorders, epilepsy, sexual dysfunction, obesity, gastroparesis, pelvic pain, interstitial cystitis, sleep apnea, neural control of prosthetic devices, or stimulation to provide peripheral sensation. A medical device delivers electrical stimulation therapy via one or more leads that include electrodes located proximate to target locations associated with the brain, the spinal cord, pelvic nerves (e.g., sacral nerve stimulation, tibial nerve stimulation, saphenous nerve stimulation, pudendal nerve stimulation, dorsal genital nerve stimulation, inferior rectal nerve, perineal nerve), peripheral nerves, or the gastrointestinal tract of a patient. For bipolar stimulation, the electrodes used for stimulation may be on one or more leads. For unipolar stimulation, the electrodes may include one or more leads and an electrode on a stimulator housing located remotely from the target site (e.g., near clavicle or near buttocks).

A clinician (or other healthcare provider) or patient may select values for a number of programmable parameters, via an external programmer, in order to define the electrical stimulation therapy to be delivered by the implantable stimulator to a patient. For example, the clinician may select one or more electrodes for delivery of the stimulation, a polarity of each selected electrode, a voltage or current pulse amplitude, a pulse width, and a pulse rate as stimulation parameters. A set of parameters, such as a set including electrode combination, electrode polarity, amplitude, pulse width, and pulse rate, may be referred to as a program in the sense that the parameters define the electrical stimulation therapy to be delivered to the patient.

### SUMMARY

This disclosure describes example devices, systems, and techniques for programming electrical stimulation based on sensed evoked signals. The techniques described herein may be useful for a variety of situations such as stimulation of nerves (e.g., sacral or pudendal nerves) that may be near muscles that may also be stimulated with the stimulation. In some examples, the system may program stimulation therapy by selecting one or more stimulation electrodes automatically and/or control a user interface to notify a clinician or other user of a recommended stimulation electrode (or information identifying differences between possible stimulation electrodes) and/or change to one or more other stimulation parameters. An implantable medical device (IMD) and/or an external programmer for the IMD may monitor one or more sensed electrical signals after implantation of the IMD.

The system may compare one or more features of the sensed electrical signals in different windows of time after the electrical stimulus. For example, a first window may be selected to include an evoked signal, if elicited by the electrical stimulus, and a beginning of a second window may occur later in time than a beginning of the first window to capture baseline electrical activity. Based on the comparison of the features of the signals within these windows, the system may identify the presence of the evoked signal and/or use the evoked signal to provide initial programming of stimulation parameters (e.g., stimulation electrode configuration, amplitude, etc.) or may improve ongoing therapy by completing one or more actions that may include adjusting one or more of the stimulation electrodes and/or control a user interface to notify a clinician or other user of a recommended change to one or more stimulation electrodes (or changes to one or more other stimulation parameters).

In one example, a system includes: processing circuitry configured to: control stimulation circuitry to deliver an electrical stimulus to a patient; control sensing circuitry to sense an electrophysiological signal after the electrical stimulus is delivered; compare a first portion of the electrical signal within a first sensing window to a second portion of the electrical signal within a second sensing window, wherein the first sensing window occurs prior to the second sensing window; identify, based on the comparison of the first portion to the second portion, an evoked response within the first sensing window that was elicited by the electrical stimulus; and determine, based on the evoked response identified from the electrical signal, a set of electrodes of a plurality of electrodes as stimulation electrodes.

In another example, a method includes: controlling, by processing circuitry, stimulation circuitry to deliver an electrical stimulus to a patient; controlling, by the processing circuitry, sensing circuitry to sense an electrophysiological signal after the electrical stimulus is delivered; comparing, by the processing circuitry, a first portion of the electrical signal within a first sensing window to a second portion of the electrical signal within a second sensing window, wherein the first sensing window occurs prior to the second sensing window; identifying, by the processing circuitry and based on the comparison of the first portion to the second portion, an evoked response within the first sensing window that was elicited by the electrical stimulus; and determining, by the processing circuitry and based on the evoked response identified from the electrical signal, a set of electrodes of a plurality of electrodes as stimulation electrodes.

In another example, a non-transitory computer-readable storage medium includes instructions that, when executed by processing circuitry, causes the processing circuitry to: control stimulation circuitry to deliver an electrical stimulus to a patient; control sensing circuitry to sense an electrophysiological signal after the electrical stimulus is delivered; compare a first portion of the electrical signal within a first sensing window to a second portion of the electrical signal within a second sensing window, wherein the first sensing window occurs prior to the second sensing window; identify, based on the comparison of the first portion to the second portion, an evoked response within the first sensing window that was elicited by the electrical stimulus; and determine, based on the evoked response identified from the electrical signal, a set of electrodes of a plurality of electrodes as stimulation electrodes.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) configured to deliver electrical stimulation to one or more pelvic nerves of a patient according to an example of the techniques of the disclosure.

FIG. 2 is a block diagram of the example IMD of FIG. 1 for delivering electrical stimulation according to an example of the techniques of the disclosure.

FIG. 3 is a block diagram of the external device of FIG. 1 for communicating with a medical device according to an example of the techniques of the disclosure.

FIG. 4 is a flowchart illustrating an example operation of a device configured to determine whether the signal of interest is present in a sensed electrical signal from any of the electrodes and then responsively adjusting stimulation electrodes.

FIG. 5 is a flowchart illustrating an example operation of a device configured to sense evoked and baseline responses for a certain number of windows and report a presence of an evoked signal based on a comparison of the evoked and baseline responses for the windows.

FIG. 6A is a flowchart illustrating an example operation of a device configured to employ alternating polarity stimuli for averaging evoked responses.

FIG. 6B is graph of an example alternating polarity stimulation employing active recharge.

FIG. 6C is graph of an example alternating polarity stimulation employing passive recharge.

FIG. 7 is a flowchart illustrating an example operation of a device configured to select a stimulation electrode combination based on a comparison of sensed signals within different windows after a delivered stimulus.

FIG. 8 is a graph of an example evoked response with an example first sensing window and an example second sensing window.

### DETAILED DESCRIPTION

This disclosure describes example devices, systems, and techniques related to determining one or more electrical parameters based on an identified evoked response. For example, the process may include selecting an electrode combination as stimulation electrodes for sacral nerve stimulation based on one or more features of an evoked response. Clinicians may select one or more electrodes based on a combination of which electrodes have the lowest impedance in view of their experience, intuition, or other information. The electrodes which deliver stimulation may not be adjusted after selection. However, the patient's response to stimulation may change after electrode selection (e.g., day to day, week to week, and month to month), and the one or more electrodes which provided suitable therapy for the patient in the beginning, may become insufficient or uncomfortable for stimulation later. Therefore, a device, method, or system capable of automatically selecting one or more electrodes as stimulation electrodes, based on an evoked response sensed over time, could reduce the burden on clinicians for identifying appropriate stimulation parameters and improve patient therapy over time.

As described herein, a medical device may deliver a test-stimulation (e.g., an electrical stimulus) and sense an electrical signal after the electrical stimulus. The sensed electrical signal may include a response evoked by the test-stimulation (if such response was indeed evoked) in a sensing window and also sense a baseline response from the electrical signal in a baseline window. These two windows may be timed such that any evoked response may occur within the sensing window (or first window), and background signals making up the baseline response or baseline activity still occurs during the baseline window (or second window). Although these windows may not overlap in time, the windows may partially overlap in some examples. In this manner, one window occurring prior to another window may indicate at least a portion of the first window occurs prior to the second window or the entire first window occurs prior to the second window. The baseline window may thus enable the system to identify background electrical activity or non-target activity that could generally be occurring due to patient movement, organ function, or other “noise” that is separate from the evoked response. The sensing window may then be used to select the portion of the sensed electrical signal that could include the evoked response.

The medical device may then compare the first portion of the electrical signal within the sensing window to the second portion of the electrical signal within the baseline window to determine if an evoked signal of interest is present within the sensed electrical signal. The medical device may repeat the sensing and comparing for one or more windows for each electrode of the one or more electrodes. In some examples, the system may compare the two windows occurring after each stimulus. In other examples, the system may re-use the baseline signal, or baseline window information, for comparison to different sensing window information where the baseline window information may be substantially similar. The medical device may, based on the comparisons, select one or more electrodes as therapy electrodes. In some examples, other parameters, such as amplitude, pulse width, frequency, etc., may be determined using this evoked signal information.

Advantages of selecting one or more electrodes as therapy electrodes based on the comparison of the signal window to the baseline window may include reducing the number of patient visits to the clinician to adjust therapy, thereby reducing medical care costs due to reduced clinician time, and improving the patient experience. Advantages of selecting one or more electrodes as therapy electrodes based on the comparison of the signal window to the baseline window additionally may include reducing the stimulation amplitude thereby decreasing power requirements and battery usage. Advantages of sensing in a sensing window and a baseline window include increasing a signal to noise ratio in the signal of interest detection as well as decrease the amplitude of stimulation suitable for screening electrodes and then select electrodes for stimulation. This process may also decrease a power requirement of stimulation as well as decrease the chance a patient feels the test-stimulation. Reducing power requirements may increase a longevity of the device and thereby decrease the frequency of replacement (i.e., explant and implant.).

To measure the response of the patient to stimulation, the medical device may sense any signal of the patient via implanted electrodes. The medical device may be an implantable medical device configured to deliver electrical stimulation via the one or more implanted electrodes. The one or more implanted electrodes may be implanted proximate a sacral nerve of the patient. The sensed signal may be a physiological response to a test stimulation. The sensed signal may be an Evoked Compound Action Potential (ECAP), an Electromyography (EMG) signal, any other signal evoked in response to the test stimulation, and/or any combination thereof.

The system may utilize a single sensed signal or calculate average sensed signals using multiple sensing windows for respective electrical stimuli. A sensed signal window may be shorter than and preceding a baseline window. In some examples, 10 to 20 different electrical stimuli may be delivered and a corresponding electrical signal with respective windows may be sensed after each stimuli. Therefore, the sensed signals may include 10 to 20 sensing windows and the sensed signals may be paired with, or otherwise compared to, 10 to 20 respective baseline windows. In some examples, stimulation may be delivered and after a delay, the implantable medical device may record 30 ms, or longer, from the selected electrode. The recording may be divided into the sensing window and the baseline window. The process may be repeated for a suitable number of windows. The suitable number of sensed windows may be averaged, the suitable number of baseline windows may be averaged, and based on the averaged windows, the implantable medical device may determine whether the signal of interest is present. If the signal of interest is not present, then an amplitude of the stimulation may be increased and the process repeated. If the signal of interest is present the implantable medical device may determine if all electrodes have been tested. If all the electrodes have not been tested the above process is repeated. If all the electrodes have been tested, the implantable medical device may compare the amplitude of the stimulation at which the signal of interest was present and select one or more therapy electrodes based on which electrodes have the lowest amplitude of the stimulation at which the signal of interest was present.

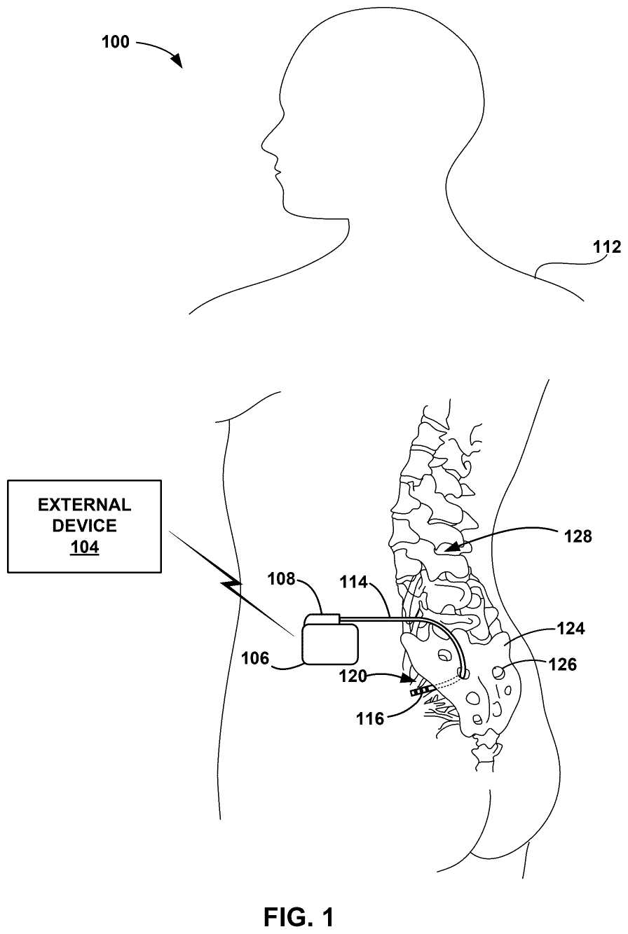

FIG. 1 is a conceptual diagram illustrating an example system 100 that includes implantable medical device (IMD) 106 which may deliver therapy to and/or sense physiological signals from target tissue. The target tissue may include or be near spinal cord 128 and/or pelvic nerves 120 (e.g., a sacral nerve or a pudendal nerve), dorsal genital nerve, perineal nerve, inferior rectal nerve, pudendal nerve, external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, detrusor muscle, or any other muscles, or any other nervous or muscle tissue that may be stimulated or from which physiological signals may be sensed of patient 112 through lead 114 (coupled to IMD 106 via connector 108). Lead 114 may carry a plurality of electrodes 116 at the distal end of lead 114. IMD 106 may provide neurostimulation to treat symptoms of patient 112, such as pain, fecal or urinary incontinence, erectile dysfunction, or other sexual dysfunction. IMD 106 may thus be configured to provide sacral nerve stimulation in one example.

In other examples, IMD 106 may be configured to deliver electrical stimulation to other nerves that may alleviate symptoms related to pelvic floor disorders. In one example, IMD 106 may be configured to deliver electrical stimulation to the tibial nerve (e.g., tibial nerve stimulation). Electrodes 116 may be implanted near a suitable portion of the tibial nerve, which may be located in a leg and/or ankle of patient 112. Lead 114 may include one or more additional leads that may carry electrodes 118 (not shown in FIG. 1). Electrodes 118 may be substantially similar to electrodes 116. In some examples, some of electrodes 116, 118 are configured to sense signals and others of electrodes 116, 118 may be configured to deliver adaptive electrical stimulation to the target tissue. In other examples, all of electrodes 116, 118 are configured to both sense signals and deliver adaptive electrical stimulation to nerve 120. In some examples, unipolar stimulation is possible where one electrode is a housing or otherwise on the housing (i.e., a can) of IMD 106.

Although the examples described in this disclosure are generally applicable to a variety of medical devices including external devices and IMDs, application of such techniques to IMDs and, more particularly, implantable electrical stimulators (e.g., neurostimulators) are described for purposes of illustration. More particularly, the disclosure will refer to a sacral nerve stimulation (SNS) for purposes of illustration, but without limitation as to other types of medical devices or other therapeutic applications of stimulation. Specifically, although FIG. 1 is directed to sacral nerve stimulation (SNS), e.g., to treat chronic pain, bladder dysfunction (e.g., retention, overactive bladder, urgency, urgency frequency, urinary incontinence, bladder incontinence, stress incontinence, nocturia, or any other dysfunction of the bladder), bowel dysfunction (e.g., fecal incontinence, intractable constipation, irritable bowel syndrome, inflammatory bowel disease, or any other dysfunction of the bowels), erectile dysfunction, or other sexual dysfunction, system 100 may be configured to treat any other condition that may benefit from electrical stimulation therapy such as interstitial cystitis, tremor, Parkinson's disease, multiple sclerosis, other movement disorders, seizure disorders (e.g., epilepsy). For example, system 100 may be configured to deliver SCS therapy, e.g., used to treat pain, and/or system 100 may be configured to deliver DBS therapy, e.g., used to treat obesity, gastroparesis, sleep apnea, or psychiatric disorders (e.g., depression, mania, obsessive compulsive disorder, anxiety disorders, and the like). In this manner, system 100 may be configured to deliver one or more of sacral nerve stimulation (SNS), deep brain stimulation (DBS), spinal cord stimulation (SCS), tibial nerve stimulation (TNS), saphenous nerve stimulation, pelvic stimulation (e.g., pudendal nerve stimulation, dorsal genital nerve stimulation, inferior rectal nerve, perineal nerve), pelvic floor stimulation, gastric stimulation, gastrointestinal stimulation, peripheral nerve field stimulation (PNFS), peripheral nerve field stimulation (PNFS), cortical stimulation (CS), or any other stimulation therapy capable of treating a condition of patient 112. Patient 112 ordinarily is a human patient. In some cases, however, therapy system 100 may be applied to other mammalian, non-mammalian, or non-human patients.

SNS, or other therapies such as DBS or TNS, may operate open loop or, alternatively, adaptive in the sense that IMD 106 may adjust, increase, or decrease the magnitude of one or more parameters of the SNS in response to changes in patient activity or movement, a severity of one or more symptoms of a disease of patient 112, a presence of one or more side effects due to the SNS, or one or more sensed signals of patient 112. For example, one example of system 100 is an SNS system with capabilities to both deliver stimulation and sense intrinsic neuronal signals. System 100 may provide for “closed-loop” therapy where IMD 106 may continuously monitor the state of certain biomarker signals and deliver stimulation according to pre-programmed routines based on the biomarker signals.

IMD 106 includes a therapy module (e.g., which may include processing circuitry, signal generation circuitry, or other electrical circuitry configured to perform the functions attributed to IMD 106) that includes a stimulation generator configured to generate and deliver electrical stimulation therapy to patient 112 via a subset of electrodes 116, 118 of lead 114. The subset of electrodes 116, 118 that are used to deliver electrical stimulation to patient 112, and, in some cases, the polarity of the subset of electrodes 116, 118 may be referred to as a stimulation electrode combination. The stimulation electrode combination may be selected for a particular patient 112 and target tissue site (e.g., selected based on the patient condition or selected based on the lead orientation with respect to nerve 120). The group of electrodes 116, 118 includes at least one electrode and may include a plurality of electrodes. In some examples, the plurality of electrodes 116, 118 have a complex electrode geometry such that two or more electrodes are located at different positions around the perimeter of the respective lead.

In some examples, sensed signals reflect changes in electrical current produced by the sum of electrical potential differences among nerves, such as nerve 120, in the region. Examples of neurological signals include, but are not limited to, bioelectric signals generated from local nerves and muscles sensed within one or more regions near nerve 120. In some examples, IMD 106 employ electroneurogram (ENG) to measure a response of the muscles near nerve 120 or innervated by nerve 120. An electroneurogram may record the electrical activity of neurons of the central nervous system (brain and spinal cord) or the peripheral nervous system (nerves and ganglions). An ENG may involve placing electrodes in or proximate neural tissue to record the electrical signals generated by neural tissue. In some examples, IMD 106 may sense evoked signals, such as evoked compound action potentials (ECAPs), evoked resonant neural activity (ERNA), electromyogram (EMG), etc. IMD may additionally sense baseline signals which are different than evoke signals. In some examples, IMD 106 may sense evoked signals in response to one or more stimulations provided by IMD 106. IMD 106 may provide stimulations which are bipolar or unipolar. IMD 106 may provide stimulations which include active recharge or passive recharge. IMD 106 may provide stimulations wherein the polarity alternates between two or more electrodes of electrodes 116, 118.

Electrical stimulation generated by IMD 106 may be configured to manage a variety of disorders and conditions. In some examples, the stimulation generator of IMD 106 is configured to generate and deliver electrical stimulation pulses to patient 112 via electrodes of a selected stimulation electrode combination. However, in other examples, the stimulation generator of IMD 106 may be configured to generate and deliver a continuous wave signal, e.g., a sine wave or triangle wave. In either case, a stimulation generator within IMD 106 may generate the electrical stimulation therapy for SNS according to a selected therapy program. In examples in which IMD 106 delivers electrical stimulation in the form of stimulation pulses, a therapy program may include a set of therapy parameter values (e.g., stimulation parameters), such as a stimulation electrode combination for delivering stimulation to patient 112, pulse frequency, pulse width, and a current or voltage amplitude of the pulses. As previously indicated, the electrode combination may indicate the specific electrodes 116, 118 that are selected to deliver stimulation signals to tissue of patient 112 and the respective polarities of the selected electrodes. The electrical stimulation generated by IMD 106 may generate, for example, burst pulses, interleaved pulses, or concurrent pulses.

In some examples, electrodes 116, 118 are radially-segmented electrodes. Radially-segmented electrodes refer to electrodes that are segmented radially along the lead. As one example, lead 114 may include a first set of electrodes 116, 118 arranged circumferentially around lead 114 that are all at the same height level on lead 114. Each of electrodes 116, 118 in the first set of electrodes 116, 118 is a separate segmented electrode and form a level of radially-segmented array of electrodes. Lead 114 may include a second set of electrodes arranged circumferentially around lead 114 that are all at the same height level on lead 114. Each of the electrodes 116, 118 in the first set of electrodes is a separate segmented electrode and form a level of radially-segmented array of electrodes. Segmented electrodes 116, 118 may be beneficial for directional stimulation and sensing.

IMD 106 may be implanted within a subcutaneous pocket, or at any other suitable site within patient 112. Generally, IMD 106 is constructed of a biocompatible material that resists corrosion and degradation from bodily fluids. IMD 106 may include a hermetic housing to substantially enclose components, such as a processor, therapy module, and memory.

System 100 may additionally include an implanted lead extension coupled to IMD 106 via connector 108 (also referred to as a connector block or a header of IMD 106). In the example shown in FIG. 1, lead 114 is implanted near a sacral region of the spinal cord of patient 112 in order to deliver electrical stimulation to one or more regions of nerve 120, which may be selected based on the patient condition or disorder controlled by therapy system 100. The specific target tissue site and the stimulation electrodes used to deliver stimulation to the target tissue site, however, may be selected, e.g., according to the identified patient responses (ex. toe/bellow motor response) and/or other sensed patient parameters (EMG/ECAP). For example, the target tissue site may be the location of bioelectric signals includes a signal component of interest. The stimulation electrodes used to deliver stimulation to the target tissue site may be those that produce the largest evoked response, e.g., using the example techniques described in this disclosure. Other lead 114 and IMD 106 implant sites are suitable depending on clinical application or target tissue/nerve.

Existing lead sets include axial leads carrying ring electrodes disposed at different axial positions and so-called “paddle” leads carrying planar arrays of electrodes. In some examples, lead 114 includes electrode combinations within an axial lead, a paddle lead, or among two or more different leads. Alternatively, more complex lead array geometries may be used. Electrodes 116, 118 may additionally or alternatively be cuff electrodes wrapped around the sacral (or other pelvic) nerve.

Lead 114 may be coupled to a common lead extension (not shown) wherein the common lead extension extends from connector 108 to any point closer to the implant site or region of interest, wherein one or more leads 114 may be connected to the common lead extension. The common lead extension enables IMD 106 to be implanted at a site distal to the region of interest while enabling the one or more leads 114 to extend a shorter distance than leads 114 may otherwise extend. In other examples, one or more leads 114 may be coupled to IMD 106 via separate lead extensions or directly to connector 108. Lead 114 may be positioned to deliver electrical stimulation to one or more target tissue sites within nerve 120 to manage patient symptoms associated with a movement disorder of patient 112. Lead 114 may be implanted to position electrodes 116, 118 at suitable locations of nerve 120 through respective holes in sacrum 124. Leads 114 may be placed at any location along nerve 120 such that electrodes 116, 118 are capable of providing electrical stimulation to target tissue sites along nerve 120 during treatment. For example, electrodes 116, 118 may be surgically implanted by inserting lead 114 through sacral foramina 126 of sacrum 124 of patient 112. Specifically, lead 114 may be inserted from a dorsal side of sacrum 124 through a sacral foramen to a ventral side of sacrum 124. Lead 114 may be inserted into the S3 sacral foramen such that electrodes extend along nerve 120 without manipulation of lead 114 on a ventral side of sacrum 124. Electrodes 116, 118 may be electrically coupled to IMD 106 via one or more leads 114. In some examples, lead 114 may be inserted into other foramen, such as S2 or S4.

In the example shown in FIG. 1, electrodes 116, 118 of leads 114 are shown as ring electrodes. Ring electrodes may be used in SNS applications because ring electrodes are relatively simple to program and are capable of delivering an electrical field to any tissue adjacent to electrodes 116, 118. In other examples, electrodes 116, 118 may have different configurations. For example, at least some of electrodes 116, 118 of leads 114 may have a complex electrode array geometry that is capable of producing shaped electrical fields. The complex electrode array geometry may include multiple electrodes (e.g., partial ring or segmented electrodes) around the outer perimeter of each lead 114, rather than one ring electrode. In this manner, electrical stimulation may be directed in a specific direction from leads 114 to enhance therapy efficacy and reduce possible adverse side effects from stimulating a large volume of tissue.

In some examples, a housing of IMD 106 includes one or more stimulation and/or sensing electrodes. The housing of IMD 106 may additionally or alternatively be referred to as a can. The can may operate as an electrode by providing stimulation and/or sensing through the surface of the device itself. In some examples, leads 114 have shapes other than elongated cylinders as shown in FIG. 1. For example, lead 114 may be paddle leads, spherical leads, bendable leads, or any other type of shape effective in treating patient 112 and/or minimizing invasiveness of lead 114.

IMD 106 includes a memory to store a plurality of therapy programs that each define a set of therapy parameter values. In some examples, IMD 106 selects a therapy program from the memory based on various parameters, such as sensed patient parameters and the identified patient behaviors. IMD 106 may generate electrical stimulation based on the parameters of the selected therapy program to manage the patient symptoms associated with a pelvic health disorder.

External device 104 wirelessly communicates with IMD 106 as suitable to provide or retrieve therapy information. External device 104 is an external computing device that the user, e.g., a clinician and/or patient 112, may use to communicate with IMD 106. For example, external device 104 may be a clinician programmer that the clinician (e.g., doctor, physician, nurse) uses to communicate with IMD 106 and program one or more therapy programs for IMD 106. Alternatively, external device 104 may be a patient programmer that allows patient 112 to select programs and/or view and modify therapy parameters. The clinician programmer may include more programming features than the patient programmer. In other words, more complex or sensitive tasks may only be allowed by the clinician programmer to reduce or limit an untrained patient from making changes to IMD 106. External device 104 may be any type of computing device, such as a proprietary device, a cellular phone, a smartphone, a tablet computing device, a laptop, or any other type of computing device. Generally, external device 104 includes a user interface that may provide and/or receive information from a user. In some examples, external device 104 is configured to pass information between IMD 106 and a different external device, but external device 104 may or may not have a user interface for programming. Instead, external device 104 may receive programming commands from the different external device (e.g., a server or other computing device that includes a user interface) and transmit those programming commands to IMD 106 and/or receive information from IMD 106 and send that information to the different external device.

When external device 104 is configured for use by the clinician, external device 104 is configured to transmit initial programming information to IMD 106. This initial information may include hardware information, such as the type of leads 114 and the electrode arrangement, the position of leads 114 adjacent to nerve 120, the configuration of electrodes 116, 118 (i.e., an electrode array), initial programs defining therapy parameter values, and any other information the clinician suitably programs into IMD 106. External device 104 may also be capable of completing functional tests (e.g., measuring the impedance of electrodes 116, 118 of leads 114).

The clinician may also store therapy programs within IMD 106 with the aid of external device 104. During a programming session, the clinician may determine one or more therapy programs that may provide efficacious therapy to patient 112 to address symptoms associated with the patient condition, and, in some cases, specific to one or more different patient states, such as a sleep state, movement state or rest state. For example, the clinician may select one or more stimulation electrode combinations with which stimulation is delivered to nerve 120 and such stimulation electrode combinations may change based on the one or more different patient states and additionally may be continuously or intermittently updated by IMD 106. During the programming session, the clinician may evaluate the efficacy of the specific program being evaluated based on feedback provided by patient 112 or based on one or more sensed physiological parameters of patient 112. External device 104 may assist the clinician in the creation/identification of therapy programs by providing a methodical system for identifying potentially beneficial therapy parameter values and/or identifying which electrodes 116, 118 to deliver stimulation from and/or which electrodes 116, 118 to sense signals.

However, in some examples, IMD 106 or external device 104 (e.g., a clinician programmer, a patient programmer, a recharger, a programmer fob, any electronic device suitable to install a therapy application, a smartphone, personal computing device, etc.), alone or in combination, may automatically determine electrode configuration and therapy parameters. For example, the medical device may determine which electrodes to use for stimulation based on a comparison of an evoked response to a baseline signal. In some examples, external device 104 outputs information indicating the selected electrode configuration for stimulation and the determined stimulation amplitude or other therapy parameter for the clinician (e.g., a clinician or a physician) to review and confirm before IMD 106 delivers therapy via the selected electrode configuration with the determined stimulation amplitude. In some examples, external device 104 outputs information indicating the selected electrode configuration for sensing. External device 104 or IMD 106 may additionally or alternatively automatically adjust the stimulation electrodes and/or the sensing electrodes based on one or more criteria. The one or more criteria may be preset by a clinician.

External device 104 may also be configured for use by patient 112. When configured as a patient programmer, external device 104 may have limited functionality (compared to a clinician programmer) in order to reduce or limit patient 112 from altering critical functions of IMD 106 or applications that may be detrimental to patient 112. In this manner, external device 104 may only allow patient 112 to adjust values for certain therapy parameters or set an available range of values for a particular therapy parameter. For example, external device 104 may only allow patient 112 to adjust an amplitude or an intensity (by combination or amplitude, pulse width and/or pulse rate).

External device 104 may also provide an indication to patient 112 when therapy is being delivered, when patient input has triggered a change in therapy or when the power source within external device 104 or IMD 106 needs to be replaced or recharged. For example, external device 104 may include an alert LED and/or a touchscreen, may flash a message to patient 112 via a programmer display, generate an audible sound or somatosensory cue to confirm patient input was received, e.g., to indicate a patient state or to manually modify a therapy parameter.

Therapy system 100 may be implemented to provide chronic stimulation therapy to patient 112 over the course of several months or years. However, system 100 may also be employed on a trial basis to evaluate therapy before committing to full implantation. If implemented temporarily, some elements of system 100 may not be implanted within patient 112. For example, patient 112 may be fitted with an external medical device, such as a trial stimulator, rather than IMD 106. The external medical device may be coupled to percutaneous leads or to implanted leads via a percutaneous extension. If the trial stimulator indicates SNS system 100 provides effective treatment to patient 112, the clinician may implant a chronic stimulator within patient 112 for relatively long-term treatment.

In some examples, IMD 106 is configured to provide electrical stimulation for treatment of a patient condition supplemental to medication provided to patient 112. Although some examples are described with the use of IMD 106 that provides stimulation, the techniques are not limited and the techniques may apply to examples where no stimulation is provided.

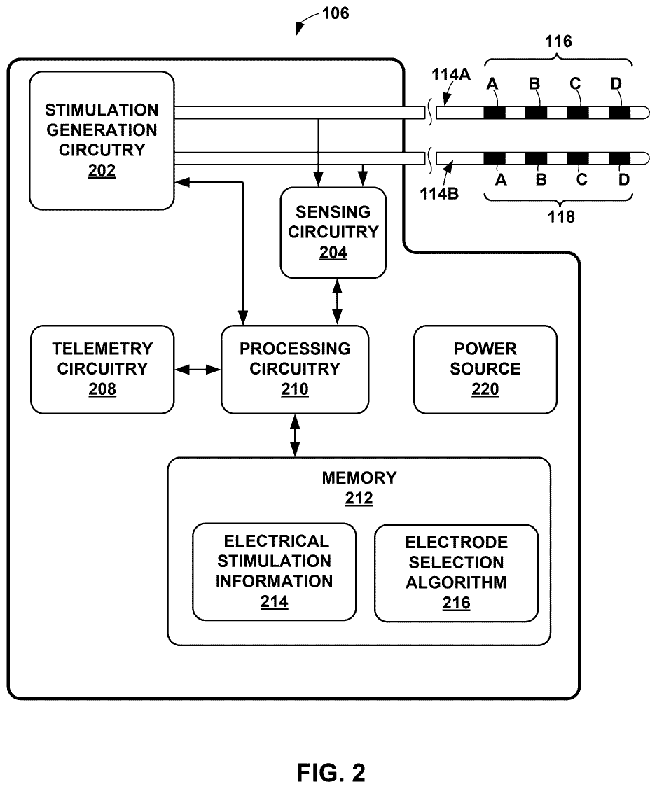

FIG. 2 is a block diagram of the example IMD 106 of FIG. 1 for delivering electrical stimulation according to an example of the techniques of the disclosure. In the example shown in FIG. 2, IMD 106 includes stimulation generation circuitry 202, sensing circuitry 204, telemetry circuitry 208, processing circuitry 210, memory 212, and power source 220. Each of these circuits may be or otherwise include electrical circuitry configured to perform the functions attributed to each respective circuit. Memory 212 may include any volatile or non-volatile media, such as a random-access memory (RAM), read only memory (ROM), non-volatile RAM (NVRAM), ferroelectric RAM (FRAM), electrically erasable programmable ROM (EEPROM), flash memory, and the like. Memory 212 may store computer-readable instructions (e.g., electrical stimulation information 214 and electrode selection algorithm 216) that when executed by processing circuitry 210, cause IMD 106 to perform various functions. Memory 212 may be a storage device or other non-transitory medium.

Stimulation generation circuitry 202 may be a single channel or multi-channel stimulation generator. In particular, stimulation generation circuitry 202 may be capable of delivering a single stimulation pulse, multiple stimulation pulses, or a continuous signal at a given time via a single electrode combination or multiple stimulation pulses at a given time via multiple electrode combinations. Stimulation generation circuitry 202 may include multiple voltage or current sources and sinks that are coupled to respective electrodes to drive the electrodes as cathodes or anodes simultaneously or at different times. IMD 106 may or may not utilize switch circuitry for time-interleaved multiplexing of stimulation via different electrodes. Switch circuitry may enable stimulation generation circuitry 202 to be configured to deliver multiple channels on a time-interleaved basis. Switch circuitry may serve to time divide the output of stimulation generation circuitry 202 across different electrode combinations at different times to deliver multiple programs or channels of stimulation energy to patient 112.

Processing circuitry 210 of IMD 106 may control sensing circuitry 204 to sense, via electrodes 116, 118 interposed along leads 114, one or more bioelectric signals of nerve 120 of patient 112. Processing circuitry 210 of IMD 106 may deliver, via electrodes 116, 118 (and stimulation generation circuitry 202), electrical stimulation therapy to patient 112 based on the sensed one or more bioelectric signals of nerve 120. Processing circuitry 210 of IMD 106 may select which electrodes of electrodes 116, 118 to connect to stimulation generation circuitry 202 and which electrodes of electrodes 116, 118 to connect to sensing circuitry 204 based on the sensed one or more bioelectric signals of nerve 120.

Telemetry circuitry 208 supports wireless communication using one or more communication protocols (e.g., using Bluetooth™, Wi-Fi™, Near-Field Communication (NFC), Near Field Magnetic Induction (NFMI), Long Term Evolution, 5th generation (LTE/5G), or MedRadio (MICS: Medical Implant Communication Service, MEDS: Medical External Device Service, MBAD: Medical Body Area Network)) between IMD 106 and an external device 104 or another computing device under the control of processing circuitry 210. In some examples, telemetry circuitry 208 supports a telemetry frequency that corresponds to a high frequency or radio frequency, which may be a radio frequency established via Bluetooth, Wi-Fi, Near-Field Communication (NFC), 175 KHz inductive telemetry, or MICS, for example. Telemetry circuitry 208 may be configured to receive an inductive sting. Processing circuitry 210 of IMD 106 may receive, as updates to programs (e.g., at least one program parameter), values for various stimulation parameters such as magnitude and electrode combination, from external device 104 via telemetry circuitry 208. The updates to the therapy programs may be stored within electrical stimulation information 214 portion of memory 212. Telemetry circuitry 208 in IMD 106, as well as telemetry modules in other devices and systems described herein, such as external device 104, may accomplish communication by radiofrequency (RF) communication techniques (e.g., Bluetooth, Wi-Fi, Near-Field Communication (NFC), or MICS). In addition, telemetry circuitry 208 may communicate with external medical device external device 104 via proximal inductive interaction of IMD 106 with external device 104. Accordingly, telemetry circuitry 208 may send information to external device 104 on a continuous basis, at periodic intervals, or upon request from IMD 106 or external device 104.

Telemetry circuitry 208 may periodically output an advertisement packet for a connection at an advertising interval. The advertisement packet may include information on how to connect with the advertising device, such as, for example, one or more of: (1) media access control (MAC) addresses for the medical device and external device; (2) a real time-point in time for the transfer to start; (3) an indication of a starting frequency; (4) an indication of a hop set; (5) a connection interval; or (6) a connection latency. Telemetry circuitry 208 may be configured to output an advertisement packet, such as, an advertisement for a wireless communication session or advertisement compliant with another protocol.

Processing circuitry 210 may include fixed function processing circuitry and/or programmable processing circuitry, and may include, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 may control stimulation generation circuitry 202 according to electrical stimulation information 214 stored in memory 212 to apply particular stimulation parameter values specified by one or more of programs, such as voltage amplitude or current amplitude, pulse width, and/or pulse rate.

In the example shown in FIG. 2, the set of electrodes 116, 118 includes electrodes 116A, 116B, 116C, 116D, 118A, 118B, 118C, and 118D. Processing circuitry 210 may control individual voltage or current sources and sinks coupled to respective electrodes 116, 118, functioning as cathodes or anodes, to deliver stimulation signals to tissue of a patient, such as patient 112. In other examples, processing circuitry may control switch circuitry to apply the stimulation signals generated by stimulation generation circuitry 202 to selected combinations of electrodes 116, 118.

Power source 220 delivers operating power to various elements of IMD 106. Power source 220 may include a small rechargeable or non-rechargeable battery and a power generation circuit to produce the operating power. Recharging may be accomplished through proximal inductive interaction between an external charger and an inductive charging coil within IMD 106. In some examples, power consumption is small enough to allow IMD 106 to utilize patient motion and implement a kinetic energy-scavenging device to trickle charge a rechargeable battery. In other examples, traditional batteries may be used for a limited period of time.

In some examples, processing circuitry 210 continuously measures the one or more bioelectric signals in real time. In other examples, processing circuitry 210 may periodically sample the one or more bioelectric signals according to a predetermined frequency, external triggering event, or after a predetermined amount of time. In some examples, the predetermined amount of time may be once an hour, once every four hours, once every 24 hours, once a week, monthly, or any suitable duration between testing to sense changes of tissue proximate electrodes 116, 118. In some examples, processing circuitry 210 periodically samples the signal at a frequency of approximately 2-35 Kilohertz (kHz). In some examples, the sampling frequency may be 21.875 kHz, 25 kHz, 30 kHz, 20 kHz, or any other frequency suitable to record the response of tissue proximate electrodes 116, 118. In some examples, processing circuitry 210 may sample the signal and/or the waveform at any frequency suitable to determine the waveform.

In the example shown in FIG. 2, memory 212 stores electrical stimulation information 214. Electrical stimulation information 214 may include program parameters (e.g., a therapy parameter set), such as a stimulation electrode combination, electrode polarity, current or voltage amplitude, pulse width, and pulse rate. In some examples, individual therapy programs are stored as a therapy group, which defines a set of therapy programs with which stimulation may be generated during a therapy session in which stimulation therapy is delivered. The stimulation signals defined by the therapy programs of the therapy group may be delivered together on an overlapping or non-overlapping (e.g., time-interleaved) basis.

Accordingly, in some examples, stimulation generation circuitry 202 generates electrical stimulation signals in accordance with the electrical stimulation information 214 stored in memory 212. Electrical stimulation information 214 may include the electrical stimulation parameters (e.g., program parameters) noted above. Other ranges of therapy parameter values may also be useful and may depend on the target stimulation site within patient 112. While stimulation pulses are described, stimulation signals may be of any form, such as continuous-time signals (e.g., sine waves) or the like. Stimulation generation circuitry 202 is an example of therapy circuitry configured to deliver a therapy from IMD 106. Other types of therapies may additionally, or alternatively, be delivered using therapy circuitry and IMD 106.

Electrical stimulation information 214 may include a current amplitude (for a current-controlled system) or a voltage amplitude (for a voltage-controlled system), a pulse rate or frequency, and a pulse width, or a number of pulses per cycle. In examples where the electrical stimulation is delivered according to a “burst” of pulses, or a series of electrical pulses defined by an “on-time” and an “off-time,” the one or more parameters may define one or more of a number of pulses per burst, an on-time, and an off-time. Processing circuitry 210 may adjust one or more parameters defining the electrical stimulation based on one or more processes described below.

In accordance with the techniques of the disclosure, processing circuitry 210 of IMD 106 may implement electrode selection algorithm 216 stored on memory 212 to select one or more electrodes from electrodes 116, 118. Techniques described herein may including processing circuitry 210 of IMD 106 implementing electrode selection algorithm 216 to select one or more electrodes, deliver a test stimulus, sense an evoked response in a first window, and sense a baseline response in a second window and compare the evoked response and the baseline response. Techniques described herein may include processing circuitry 210 of IMD 106 implementing electrode selection algorithm 216 to adjust one or more parameters of electrical stimulation information 214 based on the comparison of the baseline response and the evoked response and/or control a user interface to notify a clinician, or any other user, that the electrode selection is recommended to be programmed or adjusted. Such automatic changes to electrode selection may increase the speed with which devices are adjusted to their patients and may decrease the time clinicians spend adjusting the electrodes. An advantage of increasing the number of windows, i.e., the number of data points, is the comparison accuracy increases. Such increases in accuracy may be obtained without significantly affecting power consumption by putting an upper limit on the number of evoked response windows tested and comparing each to a respective baseline window using a peak to peak average.

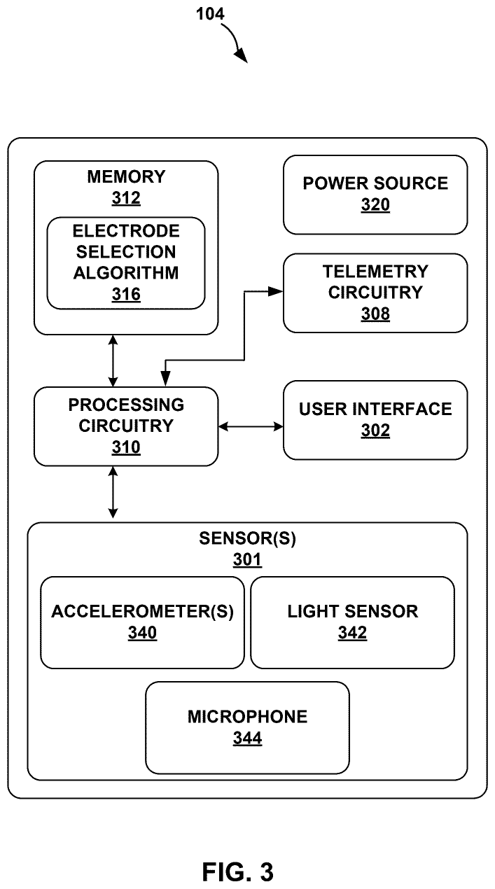

FIG. 3 is a block diagram of the external device 104 of FIG. 1. Although external device 104 may generally be described as a hand-held device, external device 104 may be a larger portable device or a stationary device. In addition, in other examples, external device 104 may be included as part of an external charging device or include the functionality of an external charging device. As illustrated in FIG. 3, external device 104 may include processing circuitry 310, memory 312, user interface 302, telemetry circuitry 308, and power source 320. Memory 312 may store instructions that, when executed by processing circuitry 310, cause processing circuitry 310 and external device 104 to provide the functionality ascribed to external device 104 throughout this disclosure. Each of these components, or modules, may include electrical circuitry that is configured to perform some or all of the functionality described herein. For example, processing circuitry 310 may include processing circuitry configured to perform the processes discussed with respect to processing circuitry.

In general, external device 104 includes any suitable arrangement of hardware, alone or in combination with software and/or firmware, to perform the techniques attributed to external device 104, and processing circuitry 310, user interface 302, and telemetry circuitry 208 of IMD 106. In various examples, external device 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. External device 104 also, in various examples, may include a memory 312, such as RAM, ROM, PROM, EPROM, EEPROM, flash memory, a hard disk, a CD-ROM, comprising executable instructions for causing the one or more processors to perform the actions attributed to them. Moreover, although processing circuitry 310 and telemetry circuitry 308 are described as separate modules, in some examples, processing circuitry 310 and telemetry circuitry 308 are functionally integrated with one another. In some examples, processing circuitry 310 and telemetry circuitry 308 correspond to individual hardware units, such as ASICs, DSPs, FPGAs, or other hardware units. While telemetry circuitry 308 is described as being arranged within external device 104, in some examples, aspects of telemetry circuitry 308 (e.g., configuring a medical device to advertise at an advertising interval or initiating a communication session) may be performed by telemetry circuitry 308 external to external device 104 (e.g., in an intermediate device).

Memory 312 (e.g., a storage device) may store instructions that, when executed by processing circuitry 310, cause processing circuitry 310 and external device 104 to provide the functionality ascribed to external device 104 throughout this disclosure. For example, memory 312 may include instructions that cause processing circuitry 310 to obtain a parameter set from memory or receive a user input and send a corresponding command to IMD 106, or instructions for any other functionality. In addition, memory 312 may include a plurality of programs, where each program includes a parameter set that defines stimulation therapy. Memory 312 may store electrode selection algorithm 316 that, when executed by processing circuitry 310, cause processing circuitry 310 and external device 104 to perform one or more of the analyses, adjustments, changes, notifications, or other processes as described with regard to the below figures.

User interface 302 may include a button or keypad, lights, a speaker for voice commands, a display, such as a liquid crystal (LCD), light-emitting diode (LED), or organic light-emitting diode (OLED). In some examples the display is a touch screen. User interface 302 may be configured to display any information related to the delivery of stimulation therapy, identified patient behaviors, sensed patient parameter values, patient behavior criteria, or any other such information. User interface 302 may also receive user input. The input may be, for example, in the form of pressing a button on a keypad or selecting an icon from a touch screen. In some examples, the touch screen may be a touch screen of a user's own device (e.g., a smartphone, tablet, computer, etc.) with a therapy application.

One or more sensors 301 may include one or more accelerometers 340, light sensor 342, and microphone 344. For example, one or more accelerometers 340 may be configured to determine information indicating a movement of external device 104. Information may include one or more of an acceleration in an x-direction, acceleration in a y-direction, or an acceleration in a z-direction. The x-direction may be perpendicular to both the y-direction and the z-direction. External device 104 may additionally, or alternatively, include a gyroscope that may detect the movement and/or generate motion information. Light sensor 342 may be configured to determine light information (e.g., an ambient light level of an environment detected by light sensor 342). Microphone 344 may be configured to determine sound information (e.g., an ambient sound level of an environment detected by microphone 344). For example, microphone 344 may detect speech (e.g., from patient 112 or a caretaker of patient 112) and/or may detect bathroom usage.

Telemetry circuitry 308 may support wireless communication between IMD 106 and external device 104 under the control of processing circuitry 310. Telemetry circuitry 308 may also be configured to communicate with another computing device via wireless communication techniques, or direct communication through a wired connection. In some examples, telemetry circuitry 308 provides wireless communication via an RF or proximal inductive medium. In some examples, telemetry circuitry 308 includes an antenna (e.g., an internal or external antenna).

Examples of local wireless communication techniques that may be employed to facilitate communication between external device 104 and IMD 106 include RF communication according to the 802.11 or Bluetooth specification sets (e.g., Classic Bluetooth, Bluetooth high speed and Bluetooth Low Energy (BLE) protocols) or other standard or proprietary telemetry protocols. In this manner, other external devices may be capable of communicating with external device 104 without needing to establish a secure wireless connection. While examples described herein may refer to connections as using the Bluetooth protocol for establishing a connection and using advertisements compliant with the Bluetooth protocol other known and future protocols may be used. For example, techniques described herein for establishing a connection between IMD 106 and external device 104 may be compliant with any RF communication protocol and/or may use any telemetry frequency.

Telemetry circuitry 308 may receive the advertisement packet from IMD 106, for example, and connect with another device (e.g., IMD 106) using the received advertisement packet. The advertisement packet may include information on how to connect with the advertising device, such as, for example, one or more of: (1) media access control (MAC) addresses for the medical device and external device; (2) a real time-point in time for the transfer to start; (3) an indication of a starting frequency; (4) an indication of a hop set; (5) a connection interval; or (6) a connection latency.

In some examples, processing circuitry 310 defines the parameters of electrical stimulation therapy, stored in memory 312, for delivering adaptive SNS to patient 112. In one example, processing circuitry 310 of external device 104, via telemetry circuitry 308, issues commands to IMD 106 causing IMD 106 to deliver electrical stimulation therapy via electrodes 116, 118 via leads 114.

In accordance with the techniques of the disclosure, processing circuitry 310 of external device 104 may implement electrode selection algorithm 316 stored on memory 312 to adjust one or more electrical stimulation parameters. Techniques described herein may include processing circuitry 310 of external device 104 implementing electrode selection algorithm 316 to control IMD 106 to select one or more electrodes, deliver a test stimulus, sense an evoked response, sense a baseline response, compare the evoked response and the baseline response for that time window and repeat the process for X additional windows, Y additional stimulation amplitude, and Z additional electrode configurations, where X, Y, and Z are integers selected to suitably test a suitable number of parameters for determining electrode-patient interface characteristics. Techniques described herein may include processing circuitry 310 of external device 104 implementing electrode selection algorithm 316 to control IMD 106 to select one or more electrodes as stimulation electrodes based on the comparison of the baseline response and the evoked response for the X windows and/or control user interface 302 to notify a clinician, or any other user, that one or more electrodes that is recommended to be adjusted. Such automatic adjustments may increase the speed with which devices are adjusted to their patients and may decrease the time clinicians spend adjusting the electrodes and selecting suitable simulation amplitude(s). An advantage of increasing the number of windows, i.e., the number of data points, is the comparison accuracy increases. Such increases in accuracy may be obtained without significantly affecting power consumption by putting an upper limit on the number of evoked response windows tested and comparing each to a respective baseline window using any number of different comparison metrics, such as a peak to peak average, area under the curve, etc.

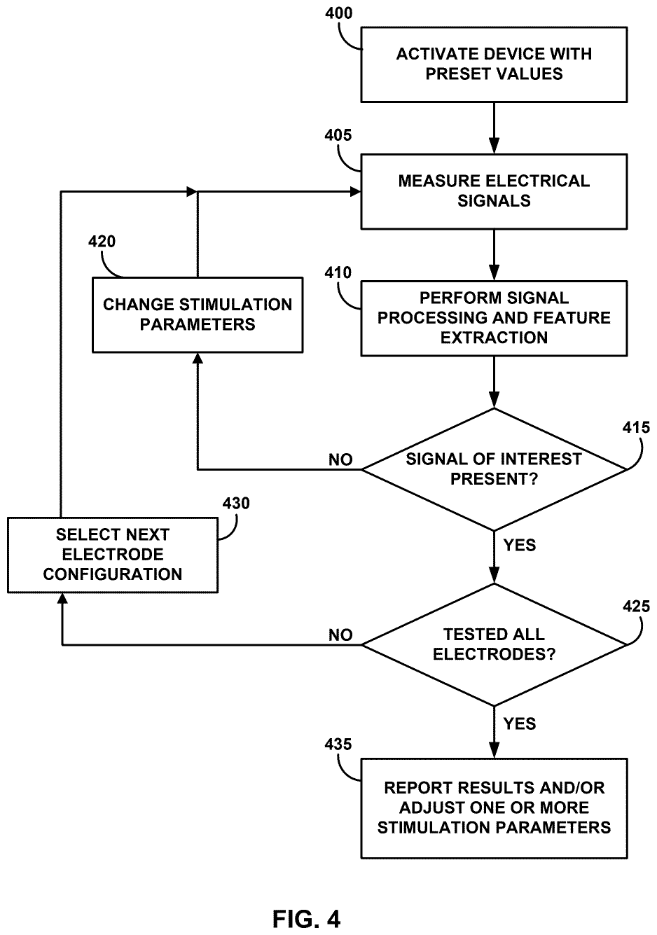

FIG. 4 is a flowchart illustrating an example operation of a device configured to determine whether the signal of interest is present in any of the electrodes and adjusting stimulation electrodes in response. The example process of FIG. 4 will be described with respect to processing circuitry 210 of IMD 106, but other processing circuitry, such as processing circuitry 310 of external device 104 may additionally, or alternatively, perform at least some of the elements of the process.

Processing circuitry 210 may, as a result of a selection by a clinician or may do so automatically, active IMD 106 with one or more values, which may be preset values (400). Processing circuitry 210 of IMD 106 may control sensing circuitry 204 to measure one or more electrical signals (405) sensed from one or more electrodes 114, 116, or 118 and/or a can of IMD 106. Processing circuitry 210 of IMD 106 may perform signal processing and feature extraction (410) on the one or more sensed electrical signals. Processing circuitry 210 of IMD 106 may determine whether there are signals of interest present (415). Based on one or more signals of interest not being present, processing circuitry 210 may increase an amplitude of stimulation (420) delivered by stimulation generation circuitry 202. Based on one or more signals of interest being present, processing circuitry 210 may determine whether all of the electrodes have been tested (425). Based on fewer than all of the suitable electrodes being tested, processing circuitry 210 may select the next electrode configuration (430) and repeat steps 405-425 for the next electrode configuration. Based on all of the electrodes having been tested, processing circuitry 210 may report results of the testing and/or adjust one or more stimulation parameters (435).

Processing circuitry 210 may, as a result of a selection by a clinician or may do so automatically, active IMD 106 with one or more values, which may be preset values (400). Activation of the device may remove the device from a hibernation or off mode, such as the modes entered into for shipping and/or storage, in order to be ready for implantation in a patient. The pre-set values may be one or more values set by a manufacturer of the device, may be one or more values pre-set by a clinician, or may be values pre-set by any one or more other entities and may be individualized to the patient and/or the patient's condition. The pre-set values may control a time active, a time asleep, stimulation parameters, and/or any other suitable parameters which may be controllable on IMD 106.

Processing circuitry 210 of IMD 106 may control sensing circuitry 204 to measure one or more electrical signals (405) sensed from one or more electrodes 114. Specifically, processing circuitry 210 may control sensing circuitry 204 to record any one or more signals from any one or more electrodes 114, 116, 118 suitable for recording whether an electrode is capable of eliciting a signal of interest from any one or more stimulation locations. In some examples, processing circuitry 210 controls sensing circuitry 204 to sense the evoked response to each of the one or more stimulation pulses during one or more respective first windows. Additionally, processing circuitry 210 may control sensing circuitry 204 to sense a baseline signal after sensing the evoked response to each of the one or more stimulation pulses, wherein sensing the baseline signal occurs in one or more respective second windows. The one or more stimulation pulses may have one or more stimulation parameters including stimulating electrode configuration, pulse width, frequency, interphase interval and/or amplitude. The one or more stimulation pulses may be delivered via electrodes 116/118. Processing circuitry 210 may control sensing circuitry 204 to record any one or more signals based on one or more sensing parameters of interest, including sensing electrode configuration, blanking parameters, evoked response window (i.e., time after a stimulation pulse when the evoked response is expected to occur), and baseline window (i.e., time when an evoked response is not expected to occur to measure the baseline noise of the system). Baseline windows may be acquired via electrodes 116/118.

Processing circuitry 210 of IMD 106 may perform signal processing and feature extraction (410) on the one or more sensed electrical signals. One or more characteristics of the sensed electrical signals may include a signal peak, a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley, a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve chronaxie, a signal strength duration curve rheobase, or another signal strength duration curve feature, a signal maximum rate of change feature (e.g., maximum of the derivative of the signal), or a signal minimum rate of change feature (e.g., the minimum of the derivative of the signal), or any other suitable signal feature.

The signal processing may include analog filtering. Analog filtering may include one or more analog filters which may be implemented in hardware. The one or more analog filters may be a high-pass filter (e.g., high frequency signals are selectively not attenuated and low frequency signals are selectively attenuated), a low-pass filter (e.g., low frequency signals are selectively not attenuated and high frequency signals are selectively attenuated), a bandstop filter (e.g., a frequency band is selectively attenuated and all other frequencies are selectively not attenuated) and a bandpass filter (e.g., a frequency band is selectively not attenuated whereas other frequencies are attenuated). The recorded signals may be digitized. The recorded signals may then be digitally filtered. Digital filtering may include one or more digital filters which may be implemented on processing circuitry 210. Digital filtering may include decimation (e.g., reducing the sampling rate of a signal by retaining only a subset of the samples, which may decrease data size and computational load while preserving essential information,) interpolation (e.g., increasing the sample rate of a signal by inserting additional sample points between existing ones, which may include upsampling followed by low-pass filtering to smooth out the resulting waveform), anti-aliasing (e.g., removing high-frequency components from a signal before sampling, as such the removed frequencies may not be misinterpreted as lower frequencies, which may thereby cause distortion in the digitized signal), and derivative (e.g., may include applying a convolution with derivative kernels, which may measure the rate of change of a signal and thereby enhance features such as rapid intensity change). A derivative filter may reduce artifacts and selectively enhance an evoked signal (e.g., an EMG and/or ECAP signal) while selectively attenuating lower frequency noise such as drifting. Additionally, the derivative filter may yield a higher amplitude, i.e., a larger slope, in a window which contains an evoked signal (e.g., an EMG and/or ECAP signal) than during a baseline signal. Additional Analog and Digital filtering may be performed as suitable to increase signal to noise ratios.

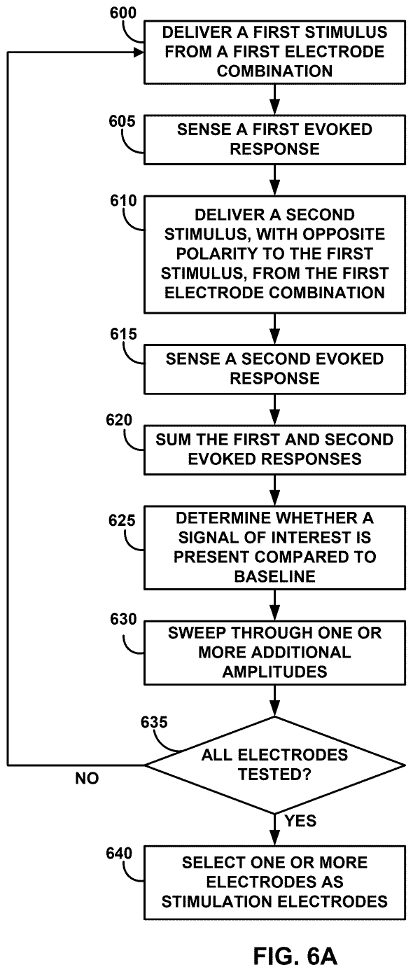

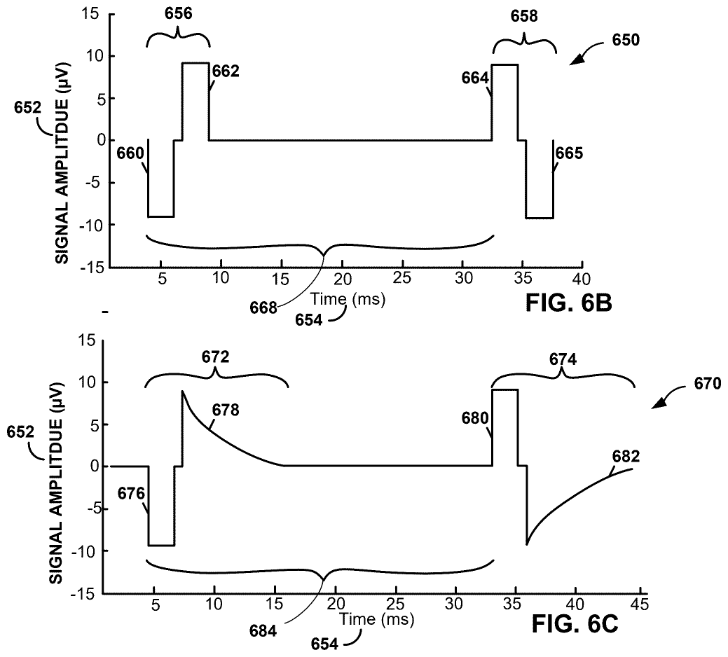

Filtering may additionally include techniques to mitigate the impact of stimulation artifact contamination. Processing circuitry 210 may perform template fitting and subtraction or summing of alternating polarity stimulation pulses. Summation of alternating polarity stimulation pulses may be substantially similar to active recharge alternating polarity stimulation 650 of FIG. 6B below and/or passive recharge alternating polarity stimulation 670 of FIG. 6C below. For example, summing of alternating stimulation pulses may include stimulation generation circuitry 202 controlling a first electrode of electrodes 116, 118 to deliver a first pulse, where the first pulse is a cathodic-leading pulse and includes an associated anode at a second electrode of electrodes 116, 118. Thereafter stimulation generation circuitry 202 may control the second electrode of electrodes 116, 118 to deliver a second pulse, where the second pulse may be a second anodic-leading pulse which has equal magnitude, where the first electrode is an associated anode. The sensed response to the first pulse may be summed with the sensed response to the second pulse. The first pulse may be delivered before the second pulse. The first electrode may be different than the second electrode. The first electrode or the second electrode may be a can of the medical device, and therefore the stimulation delivered may be monopolar stimulation. In some examples, three or more electrodes deliver the stimulation generated by the stimulation generation circuitry 202. The alternating stimulation pulses may be repeated for each of the electrodes of 116, 118.

Processing circuitry 210 may additionally mitigate other sources of noise, including electrical circuit noise, physiological noise (e.g., ECG, spontaneous myopotentials), and environmental noise (e.g., 60 Hz noise, EMI, movement artifact), through one or more analog and/or digital filters. Processing circuitry 210 may additionally mitigate outliers by determining one or more properties of a sensed window and comparing the determined one or more properties to a predetermined threshold. The one or more properties may include a power in a frequency band, a signal amplitude, and/or a peak/valley amplitude/latency. Processing circuitry 210 may determine a power in a frequency band by applying a fast Fourier transform (FFT) on a recorded signal to convert the signal from the time domain to the frequency domain. Processing circuitry 210 may then apply a bandpass filter to select a frequency band and thereafter may calculate the average of the frequency or alternatively processing circuitry 210 may calculate the power spectral density, which may include taking a squared magnitude of band passed frequency domain results. Processing circuitry 210 may compare the determined power to a threshold, wherein if the power does not meet the threshold requirements (i.e., the power of the frequency band is lower than the threshold and/or additionally or alternatively the power of a different frequency band is higher than the threshold), processing circuitry 210 may exclude the signal.

Processing circuitry 210 may calculate signal amplitude through an average of signal values to determine the signal amplitude, wherein processing circuitry 210 may exclude the signal based on the calculated signal amplitude. Processing circuitry 210 may calculate the peak to valley latency by determining a time at which the signal was at its lowest amplitude and comparing the time value at which the signal was at its highest amplitude. This comparison may yield a time between the peak and the valley of the recorded signal. Processing circuitry 210 may exclude the signal if the time between the peak and the valley is outside of an acceptable range. In some examples, the thresholds for the frequency power, the signal amplitude, and the peak/valley latency are pre-defined. In some examples, processing circuitry 210 may average one or more features over one or more windows and set the threshold for the frequency power, the signal amplitude, and the peak/valley latency based on the average over the multiple windows. Processing circuitry 210 may additionally or alternatively determine one or more population statistics of one or more features over one or more windows and set the threshold for the frequency power, the signal amplitude, and the peak/valley latency based on the population statistics. The population statistics may include a standard deviation from the mean. Processing circuitry 210 may determine if the sensed data of the window meets the criteria of an outlier, wherein processing circuitry 210 may exclude that data which meets the criteria of an outlier. Processing circuitry 210 may trigger stimulation generation circuitry 202 and sensing circuitry 204 to stimulate and thereafter record one or more additional recordings to replace the one or more data classified as an outlier.

Processing circuitry 210 may determine one or more features of interested from the processed signal data. Processing circuitry 210 may determine the one or more features on individual evoked response windows and associated baseline windows. In some examples, processing circuitry 210 determines the one or more features on an average of multiple evoked response or baseline windows. In some examples, processing circuitry 210 determines the one or more features on a sum of multiple evoked response or baseline windows. Processing circuitry 210 may calculate features such as an amplitude of the signal (e.g., peak-to-peak amplitude, amplitude of peaks and valleys, envelope, etc.), temporal properties (e.g., latency of peaks and valleys, width of peaks and valleys, phase, etc.), features from transformation of the signals (e.g., Fourier, wavelet, Hilbert, etc.), template comparison, features from dimensionality reduction techniques (e.g., principal component analysis, independent component analysis, etc.), or features from population statistics of from multiple sensed windows (e.g., standard deviation of peak-to-peak amplitude across a set of signals).

Processing circuitry 210 may determine a peak-to-peak amplitude of the signal by comparing a peak of a first signal, such as a first evoked signal response, to a peak of a signal, such as a second evoked signal response. Processing circuitry 210 may determine a peak to valley response by comparing a peak of a first signal to a valley of a first signal, wherein the first signal may be a first evoked response. Processing circuitry 210 may calculate the peak to valley by determining a time at which the signal was at its lowest amplitude and comparing the time value at which the signal was at its highest amplitude. This comparison may yield a time between the peak and the valley of the recorded signal. Processing circuitry 210 may calculate a peak (or valley) width by determining a first time at which the signal crosses a set threshold, determining a second when the signal cross the set threshold again, and comparing the first time and the second time. This comparison may yield a time width of a peak or a valley.

Processing circuitry 210 may additionally or alternatively calculate a Fourier transform of the signals (e.g., the evoked response, the baseline response, and/or the evoked response modulated by the baseline response). The Fourier transform may be the result of a fast Fourier transform. Processing circuitry 210 may determine one or more features based on a calculation of a wavelet transform of the signals. Processing circuitry 210 may determine one or more features based on a calculation of a Hilbert transform of the signals. Processing circuitry 210 may determine the one or more features using template comparison where processing circuitry may calculate the correlation of an evoked response to a one or more known signal templates. Processing circuitry 210 may subtract or otherwise modulate the evoked response based on the template to determine a response compared to the template which may be an expected response format.

Processing circuitry 210 may determine the one or more features through a machine learning or principal component analysis methods. A machine learning or artificial intelligence algorithm may operate by learning patterns from data to make predictions or decisions without being explicitly programmed. The process may begin with the collection of a training dataset, which is a large set of labeled data, wherein labelled data are points of data which were labelled by a human or another AI system such that each input data set is associated with an outcome. (e.g., data sets may be associated with desirable or undesirable implantation locations.) The training dataset may be used to train the machine learning model to learn the relationships between input features and the corresponding output labels. The model may adjust internal parameters to minimize prediction errors. This phase involves techniques such as gradient descent and backpropagation in neural networks, where the model iteratively improves prediction accuracy by comparing model predictions against the actual labels and updating parameters accordingly. The neural network may include one or more input nodes, one or more hidden nodes, and one or more output nodes.

The model may be evaluated for performance after training using unseen data. This is where a test dataset may be used to test the performance of the model. The test dataset may be a separate set of data that the model has not encountered during training. By evaluating the model on the test dataset, the model's performance in real-world scenarios may be determined. Metrics such as accuracy, precision, recall, and F1-score are commonly used to measure the model's performance. If the model performs well on the test dataset, then the model has likely successfully learned the underlying patterns in the training data and may generalize to new data. In contrast, poor performance of model may indicate issues such as overfitting, where the model has learned the training data too well, including noise and outliers, and fails to generalize. In such cases, techniques like cross-validation, regularization, or gathering more diverse training data might be employed to improve the model's robustness and performance. It is well understood that machine learning and neural networks cannot be performed in the mind of a human being due to the computation complexity and timeliness requirements of such data association tasks.

Principal component analysis may include processing circuitry 210 determining one or more principal components such as eigenvectors. Principal Component Analysis (PCA) may be a technique to reduce the dimensionality of data while preserving variability of the data. PCA may include identifying the directions (principal components) along which the data varies the most. Independent component analysis (ICA) may be used in some examples. Principal components of the recorded data may be determined by eigenvectors of a covariance matrix of the data. The corresponding eigenvalues may indicate a magnitude of variance along each of the eigenvectors. PCA may transform the original data into a new set of uncorrelated variables by projecting the data onto the eigenvectors with the largest eigenvalues, thereby simplifying the dataset while retaining its essential patterns.

Processing circuitry 210 of IMD 106 may determine whether there are signals of interest present (415). To determine whether there are signals of interest present, processing circuitry 210 may, based on the extracted features, classify the evoked signals. Such parameters or characteristics of the signal may include a signal peak, a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley, a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve chronaxie, a signal strength duration curve rheobase, or another signal strength duration curve feature, a signal maximum rate of change feature (e.g., maximum of the derivative of the signal), or a signal minimum rate of change feature (e.g., the minimum of the derivative of the signal), or any other suitable signal feature.

Processing circuitry 210 may classify the one or more signals based on a comparison of the signal to a set threshold. In some examples, processing circuitry 210 may classify the evoked signals based on a peak-to-peak amplitude of the evoked response being greater than or less than a threshold range in microvolts. Processing circuitry 210 may compare a feature of the evoked response window, such as any of the features described above, to the same feature of the baseline window to determine if a signal different from baseline noise is present. Processing circuitry 210 may identify the evoked response within the first sensing window by determining that a first feature of the first portion of the electrical signal within the first sensing window satisfies a threshold relative to a second feature of the second portion of the electrical signal within the second sensing window. Processing circuitry 210 may identify the evoked response based on the determination that the first feature is greater than the second feature by a predetermined multiplier. For example, processing circuitry 210 may identify the evoked response based on the first feature being at least 1.25 times greater than the second feature. In other examples, processing circuitry 210 may identify the evoked response based on the first feature being at least 1.5 times greater than the second feature. Processing circuitry 210 may utilize other threshold multipliers, such as 1.1 times, 1.3 times, 1.4 times, 1.6 times, 1.75 times, 2.0 times, or any other suitable multiplier to distinguish the evoked response from baseline noise detected in the second sensing window.

Processing circuitry 210 may additionally or alternatively implement a machine learning classification algorithm, which may be a different machine learning algorithm than the one described above, to, based on the extracted features, classify the evoked signals. The different machine learning algorithm may be trained and operated in a substantially similar way to the one described above, except a training set may indicate whether a signal of interest is present or not. Processing circuitry 210 may implement the machine learning classification algorithm as a regression neural network, a support vector machine, a neural network, or any other machine learning classification method. Processing circuitry 210 may train the algorithm on previous data to classify the sensed windows into predefined categories (e.g., signal is present, signal is not present, there is too much noise in the recording to classify). Processing circuitry 210 may perform the classification directly in IMD 106. Processing circuitry 210 may additionally or alternatively send sensed window data to external device 106, where processing circuitry 310 may perform classification. Processing circuitry 210 may additionally or alternatively control telemetry circuitry 208 to communicate with cloud-based resources, where the cloud based resources perform classification. Communication with cloud-based resources may reduce computational cost for processing circuitry 210 for more complex machine-learning techniques, thereby saving energy of power source 220. Alternatively, this classification may be performed directly in the IMD in hardware/firmware based on a classifier built using data in a cloud-based resource. Processing circuitry 210 may additionally or alternatively classify the one or more signals based on a comparison of any one or more of the above described features to any one or more thresholds or threshold ranges. Processing circuitry 210 may classify the signal as whether a physiological evoked response is present, an evoked response is not present, or there is inadequate information to draw a conclusion.

Processing circuitry 210 may, based on determining that a specific signal of interest is not present, i.e., that an evoked response is not present, or there is inadequate information to draw a conclusion, pass to “NO” branch of block (415). Processing circuitry 210 may, in response to “NO” branch of block (415), adjust stimulation parameters (420) and repeat steps 405, 410, and 415. Processing circuitry 210 may, in adjustment to stimulation parameters (420) collect one or more additional data (e.g., electrical signals) without modification to the stimulation parameters. Additional collection may improve a signal-to-noise ratio and enable processing circuitry 210 to make a decision. Alternatively, if processing circuitry 210 determines that the evoked response is not present, processing circuitry 210 may adjust one or more of the stimulation parameters and repeat steps 405, 410, and 415 with the new, adjusted stimulation parameters. The adjusted stimulation parameters may increase one or more stimulation parameters such as to increase an intensity of the stimulation. Specifically, processing circuitry 210 may adjust any of the stimulation parameters, which may include, for example, stimulation electrode combination, stimulation amplitude (e.g., current or voltage amplitude control), pulse frequency, pulse width, the number of pulses in a burst, the number of bursts over a duration, the pulse width of a pulse in a burst, the ON-time, the OFF-time, a pattern of pulses over a duration, or any other stimulation parameters which may be adjusted for a medical device.

Processing circuitry 210 may, based on determining that a specific signal of interest is present, proceed to “YES” branch of block of 415 where processing circuitry 210 may determine whether all of the electrodes have been tested (425). Before determining whether all electrodes have been tested, processing circuitry 210 may store the one or more stimulation parameters and the properties (i.e., features) of the evoked signal in memory 212. Processing circuitry 210 may additionally or alternatively and whether or not a specific signal of interest is present, sweep stimulation pulse widths to acquire strength-duration curves of evoked responses. Processing circuitry 210 may use the strength duration curves to determine one or more suitable evoked responses and the associated stimulation parameters which effectuated the suitable evoked response. Processing circuitry 210 may store the one or more associated stimulation parameters and the properties (i.e., features) of the evoked signal in memory 212. Processing circuitry 210 may additionally or alternatively and whether or not a specific signal of interest is present, adjust any other additional stimulation parameters (e.g., frequency) in order to find a suitable evoked response based on the extracted features. Processing circuitry 210 may store the one or more associated stimulation parameters and the properties (i.e., features) of the evoked signal in memory 212.

Processing circuitry 210 may, after storing the properties to memory, determine whether all of the electrodes have been tested. If all of the electrodes have not been tested, “NO” branch of block of (425), processing circuitry 210 may select the next electrode configuration (430) and repeat steps 405, 410, 415, 420, and 425. In some examples, there may be six electrodes in electrodes 116, 118. In other examples, there may be eight, 10, 12, 16, 20, 24, or any other number, even or odd, of electrodes suitable for delivering stimulation to one or more locations of patient 112.

If all of the electrodes have been tested, “YES” branch of block of (425), processing circuitry 210 may report results and/or adjust one or more stimulation parameters (435). Processing circuitry 210 may determine which one or more stimulation parameters to adjust based on the stimulation parameters required to get one or more signals of interest. For example, processing circuitry 210 may determine which electrodes of the one or more electrodes to select as stimulation electrodes based on the one or more associated stimulation parameters and the properties (i.e., features) of the evoked signal stored in memory 212. Specifically, processing circuitry 210 may compare the stimulation parameters, the stimulation parameters which were found to be suitable stimulation parameters and stored into memory 212 in step 415, between each of electrodes to determine which stimulation parameters have the lowest energy requirement and/or effectuate the largest stimulation results. Based on the comparison, processing circuitry 210 may select one or more electrodes to use as stimulation electrodes.

Processing circuitry 210 may automatically adjust the stimulation electrodes used. Processing circuitry 210 may additionally or alternatively adjust any one or more stimulation parameters such a polarity of each selected electrode, a voltage or current pulse amplitude, a pulse width, and a pulse rate as stimulation parameters. Alternatively, processing circuitry 210 may control telemetry circuitry 208 to send a notification to a clinician to request that the clinician updates the stimulation electrodes based on the comparison results. Processing circuitry 210 may control stimulation generation circuitry 202 to deliver stimulation through the selected or determined stimulation electrodes. Stimulation generation circuitry 202 may deliver stimulation using passive recharge stimulation.

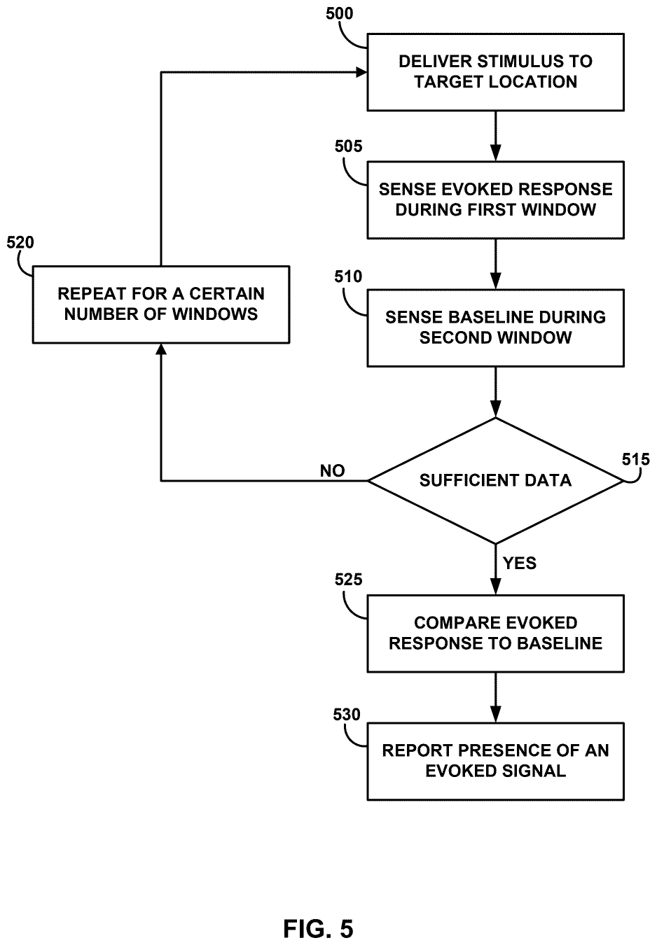

FIG. 5 is a flowchart illustrating an example operation of a device configured to sense evoked and baseline responses for a certain number of windows and select one or more stimulation electrodes based on a comparison of the evoked and baseline responses for the certain number of windows. The example process of FIG. 5 will be described with respect to processing circuitry 210 of IMD 106, but other processing circuitry, such as processing circuitry 310 of external device 104 may additionally, or alternatively, perform at least some of the elements of the process.

Processing circuitry 210 may control stimulation generation circuitry 202 to deliver one or more stimulation pulses to a target location (500). After delivering the one or more stimulation pulses, processing circuitry 210 may control sensing circuitry 204 to sense the evoked response to each of the one or more stimulation pulses during a first window (505) then processing circuitry 210 may control sensing circuitry 204 to sense baseline signals of the target location during a second window (510). In some examples, sensing circuitry 204 may sense a single signal over the first and second window, but the first portion of the signal is identified in the first window and the second portion of the signal is identified in the second window. Processing circuitry 210 may determine whether there is sufficient data (515). If there is not sufficient data “NO” branch of block of (515), processing circuitry 210 may repeat steps 500, 505, and 510 for X additional windows (520). If there is sufficient data “YES” branch of block of (510), processing circuitry 210 may analyze the evoked responses by comparing the sensed evoked responses to the sensed baseline responses using a peak to peak average (525) and thereafter processing circuitry 210 may, based on the comparison, report the presence of an evoked signal (530).

Processing circuitry 210 may control stimulation generation circuitry 202 to deliver one or more stimulation pulses to a target location (500). Processing circuitry 210 of IMD 106 may control stimulation generation circuitry 202 to deliver the one or more stimulation pulses via a plurality of electrodes 116, 118 to the target tissue. Processing circuitry 210 may control stimulation generation circuitry 202 to deliver a first pulse of a one or more stimulation pulses at a selected amplitude. Processing circuitry 210 may initiate the process of steps 500-530 at pre-set times throughout the day and/or throughout the week. The pre-set times may be scheduled for the same time each day of the week to reduce variables in comparison. For example, the scheduled pre-set time may be during sleep of patient 112, e.g., 3 am each day, such that inter-day variations are reduced.

The plurality of pulses may be delivered as active recharge alternating polarity stimulation 650 of FIG. 6B below and/or passive recharge alternating polarity stimulation 670 of FIG. 6C below. For example, the plurality of pulses may be delivered in an active recharge setting, similar to active recharge alternating polarity stimulation 650, wherein stimulation generation circuitry 202 may control a first electrode of electrodes 116, 118 to deliver a first pulse, where the first pulse is a cathodic-leading phase (of a bi-phasic pulse). Thereafter stimulation generation circuitry 202 may control a second electrode of electrodes 116, 118 to deliver a second pulse, where the second pulse may be an anodic-leading phase. Each pulse may additionally have a recharge phase of equal magnitude following each of the cathodic-leading phase and the anodic-leading phase and as such each pulse would be active recharge phases. In some examples, the stimulation generation circuitry 202 may deliver the second pulse via the second electrode before delivering the first pulse via the first electrode. During therapy delivery, for example after step 530, stimulation generation circuitry 202 may deliver a first pulse and thereafter omit the second pulse. Processing circuitry 210 may control stimulation generation circuitry 202 to passively recharge after delivering the first phase. In some examples, the first electrode may be proximate to the second electrode. In some examples, the first electrode may be distal to the second electrode.

In some examples, an amplitude of the stimulation pulses may be constant for all pulses of the one or more stimulation pulses. The amplitude of the stimulation pulses may be equal to an amplitude of the stimulation therapy which is delivered. The selected amplitude may be 0.5 mA, 0.3 mA, 0.4 mA, 0.6 mA, 0.7 mA, 1 mA, 2 mA, 5 mA, 10 mA or any other amplitude suitable to elicit one or more responses. In other examples, the selected amplitude may vary between one or more pulses of the one or more stimulation pulses. In some examples, the selected amplitude may be a stepped ramp wherein a first set of the one or more stimulation pulses has a first amplitude, a second set of the one or more stimulation pulses has a second amplitude, and one or more other sets of the one or more stimulation pulses has one or more other amplitudes. For example, the plurality of pulses may include five subsets of pulses where, a first sub-set of pulses may have a magnitude of 0.1 mA, a second subset set of pulses may have a magnitude of 0.2 mA, a third subset of pulses may have a magnitude of 0.3 mA, a fourth sub-set of pulses may have a magnitude of 0.4 mA, and a fifth sub-set of pulses may have a magnitude of 0.5 mA. The pulses may be ordered from a lowest to a highest stimulation intensity. In some examples, the pulses may be divided into any number of subsets and the stimulation amplitude may vary between and within each of the subsets. In some examples, the selected amplitude may be a ramp where each pulse of the one or more stimulation pulses has a higher amplitude than temporally preceding pulses.

Processing circuitry 210 may control sensing circuitry 204 to sense the evoked response to each of the one or more stimulation pulses during one or more respective first windows (505). Processing circuitry 210 of IMD 106 may control sensing circuitry 204 to measure electrical signals that result from the stimulation pulses at a plurality of electrodes 116, 118. The electrical signals may include electromyography (EMG) signals which may develop over the course of 10 ms. Processing circuitry 210 may control sensing circuitry 204 to sense a first stimulation-evoked signals in response to a first pulse of the one or more stimulation pulses at the selected amplitude. Processing circuitry 210 may control sensing circuitry 204 to repeat sensing for each of the one or more stimulation pulses which may be at one or more other amplitudes. Processing circuitry 210 may control sensing circuitry 204 to begin sensing immediately processing circuitry 210 controls stimulation generation circuitry 202 to deliver one or more stimulation pulses. In some examples, processing circuitry 210 may control sensing circuitry 204 to begin sensing, i.e., begin the first window, at a first delay after processing circuitry 210 controls stimulation generation circuitry 202 to deliver one or more stimulation pulses. The delay may be a set delay, such as 1 millisecond (ms), 2 ms, 3 ms, 4 ms, 5 ms, 6 ms, 7 ms, 8 ms, or any other delay after processing circuitry 210 controls stimulation generation circuitry 202 to deliver one or more stimulation pulses which is suitable for detecting the stimulation response while excluding the stimulation pulse artifacts. The first window may be 2 ms, 3 ms, 4 ms, 5 ms, 6 ms, 7 ms, 8 ms, 9 ms, 10 ms, 11 ms, 12 ms, or any other length suitable to record the evoked response.

Processing circuitry 210 may control sensing circuitry 204 to sense a baseline signal after sensing the evoked response to each of the one or more stimulation pulses, wherein sensing the baseline signal occurs in one or more respective second windows (510). Processing circuitry 210 of IMD 106 may control sensing circuitry 204 to measure electrical signals which represent a baseline response of the target location at a plurality of electrodes 116, 118. Processing circuitry 210 may control sensing circuitry 204 to sense a first baseline signal in a second window after sensing the evoked response in step 505. Processing circuitry 210 may control sensing circuitry 204 to repeat sensing of the baseline signal after each of the one or more evoked response sensing. In some examples, processing circuitry 210 controls sensing circuitry 204 to sense the baseline signal one or more times, wherein the quantity of second windows is greater than or less than the number of first windows. Processing circuitry 210 may control sensing circuitry 204 to begin sensing immediately after controlling sensing circuitry 204 to sense the evoked response. In some examples, processing circuitry 210 may control sensing circuitry 204 to begin sensing, i.e., begin the second window, at a first delay after processing circuitry 210 controls sensing circuitry 204 to sense an evoked response. The delay may be a set delay, such as 0 ms, 1 ms, 2 ms, 3 ms, 4 ms, 5 ms, 6 ms, 7 ms, 8 ms, or any other delay after processing circuitry 210 completes control of sensing circuitry 204 of sensing the evoked response. Delaying the second window after the first window may decrease the amount of noise in the data sensing circuitry 204 records which may increase a signal to noise ratio between the first window and the second window. In some examples, processing circuitry 210 may control sensing circuitry 204 to begin sensing the second window while processing circuitry 210 is controlling sensing circuitry 204 to sense an evoked response during the first window. As such, a delay between the end of the first window and the beginning of the second window may be a negative delay such as −1 ms, −2 ms, −3 ms, −4 ms, −5 ms, −6 ms, −7 ms, −8 ms, or any other negative delay where the first window overlaps with some portion of the second window. Overlapping the first window and the second window may decrease the amount of data which sensing circuitry 204 records which may decrease a power requirement of IMD 106 on power source 220. A length of the second window may be 15 ms, 16 ms, 17 ms, 18 ms, 19 ms, 20 ms, 21 ms, 22 ms, 23 ms, 24 ms, 25 ms, 26 ms, 27 ms, 28 ms, 29 ms, 30 ms, or any other length suitable to record the baseline response.

In other words, processing circuitry 210 may control stimulation generation circuitry 202 to deliver a stimulus to a target location. A first period of time later, processing circuitry 210 may control sensing circuitry 204 to record the evoked response during a first window which is a second period of time long. A third period of time later, processing circuitry 210 may control sensing circuitry 204 to record baseline signals during a second window which is a fourth period of time long. The first period of time, second period of time, third period of time, and fourth period of time may all be different periods of time or may all be the same period of time.

The first period of time may be 1 millisecond (ms), 2 ms, 3 ms, 4 ms, 5 ms, 6 ms, 7 ms, 8 ms, or any other delay after processing circuitry 210 controls stimulation generation circuitry 202 to deliver one or more stimulation pulses which is suitable for detecting the stimulation response while excluding the stimulation pulse artifacts. The second period of time may be 2 ms, 3 ms, 4 ms, 5 ms, 6 ms, 7 ms, 8 ms, 9 ms, 10 ms, 11 ms, 12 ms, or any other length suitable to record the evoked response. The third period of time may be 0 ms, 1 ms, 2 ms, 3 ms, 4 ms, 5 ms, 6 ms, 7 ms, 8 ms, or any other delay after processing circuitry 210 completes control of sensing circuitry 204 of sensing the evoked response. Delaying the third period of time after the second period of time may decrease the amount of noise in the data sensing circuitry 204 records which may increase a signal to noise ratio between the first window and the second window. In some examples, processing circuitry 210 may control sensing circuitry 204 to begin the third period of time before the end of the second period of time. As such, a delay between the end of the second period of time and a beginning of the third period of time may be a negative delay such as −1 ms, −2 ms, −3 ms, −4 ms, −5 ms, −6 ms, −7 ms, −8 ms, or any other negative delay where the first window overlaps with some portion of the second window. Overlapping the first window and the second window may decrease the amount of data which sensing circuitry 204 records which may decrease a power requirement of IMD 106 on power source 220.

The third period of time may be 15 ms, 16 ms, 17 ms, 18 ms, 19 ms, 20 ms, 21 ms, 22 ms, 23 ms, 24 ms, 25 ms, 26 ms, 27 ms, 28 ms, 29 ms, 30 ms, or any other length suitable to record the baseline response. In some examples, processing circuitry 210 may control sensing circuitry 204 to record for a set overall period of time and thereafter processing circuitry 210 may divide the set overall period of time into the first period of time, the second period of time, the third period of time, and the fourth period of time. The overall period of time may be 30 ms, 45 ms, 60 ms, or any other length suitable to record the evoked response, baseline response and suitable delays therebetween for data clarity.

Processing circuitry 210 may determine whether there is sufficient data (515). Processing circuitry 210 may determine whether there is sufficient data based on whether a sufficient number of signal sensing windows have been tested. Since processing circuitry 210 checks whether there is sufficient data prior to processing the data, processing circuitry 210 may wait until testing has completed prior to running the analysis, thereby as an advantage processing circuitry 210 may save battery. Specifically, processing circuitry 210 may save energy in an energy constrained environment by not computing analyses and later recomputing the analyses. In some examples, processing circuitry 210 is not writing the analyses to memory and storing these analyses for long periods of time which saves energy and storage space.

To determine if there is sufficient data, processing circuitry 210 may compare the number of first windows recorded to a pre-set number of first windows. The pre-set number of first windows may be 10, 12, 14, 16, 18, 20, or any suitable number of first windows which are statistically sufficient to determine whether there is a signal of interest present. Processing circuitry 210 may additionally compare the number of second windows recorded to a pre-set number of second windows. The pre-set number of second windows may be 10, 12, 14, 16, 18, 20, or any suitable number of second windows which are statistically sufficient to determine whether there is a signal of interest present. The number of second windows may be the same as or different than the number of first windows.

If there is not sufficient data “NO” branch of block of (515), processing circuitry 210 may repeat steps 500, 505, and 510 for a certain number of additional windows (520). The certain number of additional windows may be based on a comparison of the number of windows recorded and a suitable number of windows. In some examples, if the pre-set number of first windows, i.e., the suitable number of windows, is greater than the number of first windows recorded, i.e., the number of windows recorded has not yet met its threshold, processing circuitry 210 may repeat steps 500-515. In other examples, if the pre-set number of second windows, i.e., the suitable number of windows, is greater than the number of second windows recorded, i.e., the number of windows recorded has not yet met its threshold, processing circuitry 210 may repeat steps 500-515. In some examples, processing circuitry 210 may determine how many windows have been recorded and compare that to the number of windows that may be recorded and select a number of additional cycles of steps 500-510 which may bring the number of windows that have been recorded up to and equal with the number of windows that may be recorded. In some examples, whether sufficient data has been collected may be based on a passage of time since a first stimulus was delivered.

If there is sufficient data, i.e., “YES” branch of block of (515), processing circuitry 210 may then compare the evoked responses to the baseline responses using a peak to peak average (525) and thereafter processing circuitry 210 may report the presence of an evoked signal (530) based on the comparison.

Comparing one or more evoked responses to one or more baseline responses using (525) may include processing circuitry 210 analyzing the responses in any combination of one or more ways. In general, processing circuitry 210 may identify one or more features of the sensed electrical signals to characterize the sensed electrical signal according to the one or more features. In this manner, the one or more features may include peak amplitudes, peak-to-peak amplitudes, latency, etc. In some examples, comparison includes processing circuitry 210 time aligning all of the evoked responses sensed by the sensing circuitry 204 in (505) based on a peak negative portion of their response. Processing circuitry 210 may then time align all of the baseline responses sensed by the sensing circuitry 204 in (510) based on a peak negative portion of their response. Processing circuitry 210 may piecewise compare each evoked response to the baseline response, determining a difference between the evoked response to the baseline at the most negative or most positive portions of the evoked response. Based on the piecewise comparison, processing circuitry 210 may average the differences. Processing circuitry 210 may additionally compare the averaged differences to a threshold. Alternatively, processing circuitry 210 may average all of the time-aligned evoked response and then average all of the time-aligned baseline responses and compare the averaged evoked response and the averaged baseline response to determine an averaged overall difference. Processing circuitry 210 may additionally compare the averaged overall difference to a threshold.

Processing circuitry 210 may then report the presence of the evoked signal (530) based on the comparison. In some examples, processing circuitry 210 report the presence of the evoked signal to step 415 of FIG. 4, and therefore the process of FIG. 5 may fit into steps 405-415 of FIG. 4. Processing circuitry 210 may compare the averaged differences and/or the averaged overall difference between each of the electrodes. The electrodes with the highest averaged differences and/or the averaged overall difference may be reported as having the presence of the evoked signal. Having the highest averaged differences and/or the averaged overall difference may indicate that the electrodes are closer to the nerve of interest, or are otherwise superior at nerve activation compared to the other tested electrodes.

FIG. 6A is a flowchart illustrating an example operation of a device configured to employ alternating polarity stimuli for averaging evoked responses. The example process of FIG. 6A will be described with respect to processing circuitry 210 of IMD 106, but other processing circuitry, such as processing circuitry 310 of external device 104 may additionally, or alternatively, perform at least some of the elements of the process.

Processing circuitry 210 may control signal generation circuitry 202 to deliver a cathodic-leading pulse from a first electrode combination (600), control sensing circuitry 204 to sense a first evoked response (605), control signal generation circuitry 202 to deliver an anodic-leading pulse from the first electrode combination (610), control sensing circuitry 204 to sense a second evoked response (615), sum the first evoked response and the second evoked response (620), determine whether a signal of interest is present compared to baseline (625), sweep through one or more additional amplitudes (630), determine whether all electrodes have been tested (635). Based on fewer than all of the electrodes being tested, processing circuitry 210 may repeat steps 600-630. Based on all of the electrodes being tested, processing circuitry 210 may select one or more electrodes as stimulation electrodes (640).

Processing circuitry 210 may control signal generation circuitry 202 to deliver a cathodic-leading pulse from the first electrode combination (600). The first electrode combination may be a first electrode and a second electrode of any electrode of electrodes 116, 118. The cathodic-leading pulse may have a cathodic phase where a voltage of the pulse is negative. The cathodic phase may elicit a response from the target tissue proximate electrodes 116, 118. The response may be an electromyography (EMG) response. For the cathodic phase, the first electrode may be a cathode and may have an associated anode at a second electrode of electrodes 116, 118. Optionally, processing circuitry 210 may control signal generation circuitry 202 to deliver an anodic phase of the cathodic-leading pulse via the second electrode of electrodes 116, 118, where the anodic phase may be equal and opposite to the cathodic phase to perform an active recharge. Processing circuitry 210 may control signal generation circuitry 202 to deliver the anodic phase via the first electrode as an anode and the second electrode as a cathode. As such, the cathodic phase may be an active recharge pulse. In other examples, processing circuitry 210 may control signal generation circuitry 202 to receive the charge at first electrode and second electrode without driving any charge and therefore may be a passive recharge. The cathodic-leading pulse may be similar to first pulse 656 of FIG. 6B or first pulse 672 of FIG. 6C. Processing circuitry 210 may then control sensing circuitry 204 to sense a first evoked response from the second electrode (605). The first electrode may be different than the second electrode. The first electrode or the second electrode may be a can of the medical device, and therefore the stimulation delivered may be monopolar stimulation. The first electrode and the second electrode may be proximate each other.

Processing circuitry 210 may control signal generation circuitry 202 to deliver an anodic-leading pulse from the first electrode combination (610). The anodic-leading pulse may have a first phase with an opposite polarity than a first phase of the cathodic-leading pulse. In some examples, the cathodic-leading pulse may be delivered first and the anodic-leading pulse delivered second, as illustrated in FIGS. 6B and 6C. The anodic-leading pulse may elicit a response from the target tissue proximate electrodes 116, 118. The response may be an electromyography (EMG) response. A first phase of the anodic-leading pulse may be an anodic phase. For the anodic phase, the first electrode may be an anode and may have an associated cathode at a second electrode of electrodes 116, 118. Optionally, processing circuitry 210 may control signal generation circuitry 202 to deliver a second phase of the anodic-leading pulse, where the second phase may be an cathodic phase. The second phase may follow the first phase. Processing circuitry 210 may control signal generation circuitry 202 to deliver the cathodic pulse via the second electrode of electrodes 116, 118, where the cathodic phase may be equal and opposite to the anodic phase to perform an active recharge. In other examples, processing circuitry 210 may control signal generation circuitry 202 to receive the charge at first electrode and second electrode without driving any charge and therefore may be a passive recharge phase. The anodic-leading pulse may be similar to second pulse 658 of FIG. 6B or first pulse 674 of FIG. 6C. Processing circuitry 210 may then control sensing circuitry 204 to sense a second evoked response from the first electrode (615). The first electrode or the second electrode may be a can of the medical device, and therefore the stimulation delivered may be monopolar stimulation.

Processing circuitry 210 may sum the first evoked response and the second evoked response (620). In some examples, processing circuitry 210 time-aligns and sums the first evoked response and the second evoked response. Time-aligning the signals may involve time aligning the most negative portion of the first evoked response and the second evoked response. In some examples, time-aligning the signals involves time aligning the most positive portion of the first evoked response and the second evoked response. Summing the first and second pulse may increase a signal to noise ratio compared with recording from only the first electrode or recording only from the second electrode. In other examples, processing circuitry 210 may time-align and average the first evoked response and the second evoked response. In some examples, with the summing or the averaging, the first evoked response and the second evoked response may be weighted wherein either the first evoked response or the second evoked response is given precedence over the other, wherein the ratio between weighting of the first evoked response may be 75% first evoked response and 25% second evoked response.

After summation, processing circuitry 210 may determine whether a signal of interest is present compared to a baseline signal (625) and processing circuitry 210 may thereafter sweep through one or more additional amplitudes (630). In some examples, determining whether a signal of interest is present may be similar to steps 405-430 of FIG. 4, and/or steps 500-530 of FIG. 5. Specifically, processing circuitry 210 may perform any of the processing steps described above on the signal averaged from the first and second evoked responses in order to determine which electrodes of the one or more electrodes to select as stimulation electrodes. In an example, processing circuitry 210 may compare the signal averaged from the first and second evoked responses to a baseline signal recorded before or after the first and second evoked response. Thereafter processing circuitry 210 may perform any one of signal processing and feature extraction steps of step 410 of FIG. 4 on the compared signal, wherein processing circuitry 210 may determine if a signal of interest is present as done in step 415 of FIG. 4.Processing circuitry 210 may sweep through one or more additional amplitudes (630) by repeating steps 600-625 of FIG. 6A with a different amplitude for the first stimulus and/or the second stimulus. In some examples, the one or more amplitudes may ramp up between each consecutive stimulus until a trigger condition, such as the presence of a signal of interest and/or the one or more amplitudes may ramp up based on a pre-set schedule. In some examples, processing circuitry 210 may alter any one or more other parameters of the first stimulus or the second stimulus such as electrode combination, electrode polarity, amplitude, pulse width, and pulse rate. In some examples, processing circuitry 210 may sweep through one or more other parameters based on whether a signal of interest is present compared to a baseline signal. For example, processing circuitry 210 may increase an amplitude of the first stimulus and the second stimulus in response to a determination that the signal of interest is not present compared to the baseline signal.

Processing circuitry 210 determine whether all electrodes have been tested (635). Based on fewer than all of the electrodes being tested “NO” branch of block of (635), processing circuitry 210 may repeat steps 600-630. Based on all of the electrodes being tested “YES” branch of block of (635), processing circuitry 210 may select one or more electrodes as stimulation electrodes (640). Processing circuitry 210 may select the one or more electrodes as stimulation electrodes. (640) Processing circuitry 210 may automatically adjust the stimulation electrodes used. Alternatively, processing circuitry 210 may control telemetry circuitry 208 to send a notification to a clinician to request that the clinician updates the stimulation electrodes based on the comparison results.

FIG. 6B is graph of an example alternating polarity stimulation employing active recharge. FIG. 6C is graph of an example alternating polarity stimulation employing passive recharge. FIGS. 6B and 6C will be discussed together. The example alternating polarity stimulations of FIGS. 6B and 6C will be described with respect to processing circuitry 210 and related components of IMD 106, but other processing circuitry and related components, such as processing circuitry 310 of external device 104 may additionally, or alternatively, be employed. Active recharge alternating polarity stimulation 650 and passive recharge alternating polarity stimulation 670 illustrate examples of stimulation that processing circuitry 210 may control stimulation generation circuitry 202 to deliver through electrodes 116, 118 of IMD 106. Signal amplitude 652 is measured in microvolts (μV). Time axis 654 represents a time over which the signals are delivered.

Active recharge alternating polarity stimulation 650 may include first pulse 656 and second pulse 658. In some examples, active recharge alternating polarity stimulation 650 may include any number of pulses suitable to deliver a test stimulation to a patient. In some examples, active recharge alternating polarity stimulation 650 includes three, four, five, six, or any other suitable number of pulses to determine whether a signal of interest is present. First pulse 656 may include first cathodic phase 660 and first anodic phase 662 separated by an interphase interval. Processing circuitry 210 may control stimulation generation circuitry 202 to deliver first cathodic phase 660 as pulse of negative amplitude for a first duration, pause for a period of time shorter than the first duration, then processing circuitry 210 may control stimulation generation circuitry 202 to deliver first anodic phase 662 of positive amplitude for the first duration or for a second duration suitable to reduce the built up charge from first cathodic phase 660. In some examples, the area under the curve, i.e., the amount of charge delivered, by first cathodic phase 660 and first anodic phase 662 may be equal. Processing circuitry 210 may control sensing circuitry 204 to record from electrode 116, 118 after controlling stimulation generation circuitry 202 to deliver first anodic phase 662. Processing circuitry 210 may wait for a period such as period 668. Period 668 may be the inverse of the frequency at which pulses are delivered. In some examples, the frequency may be 2-25 kHz or any other frequency suitable to deliver one or more test pulses.

After period 668, processing circuitry 210 may deliver second pulse 658. Second pulse 658 may include second anodic phase 664 and second cathodic phase 665 separated by an interphase interval. To deliver second pulse 658, processing circuitry 210 may control stimulation generation circuitry 202 to deliver second anodic phase 664 as pulse of positive amplitude for a first duration, pause for a period of time shorter than the first duration, then processing circuitry 210 may control stimulation generation circuitry 202 to deliver second cathodic phase 665 of negative amplitude for the first duration or for a second duration suitable to reduce the built up charge from first cathodic phase 660. In some examples, the area under the curve, i.e., the amount of charge delivered, by second cathodic phase 664 and second anodic phase 665 may be equal. In other examples, first pulse 656 and second pulse 658 may be flipped. In other examples, first pulse 656, or second pulse 658, may be repeated one or more additional times. In some examples, first pulse 656 and second pulse 658 may both be anodic-leading pulses (i.e., both 660 and 664 are anodic phases with 662 and 665 being cathodic phases) or may both be cathodic-leading pulses (i.e., both 660 and 664 are cathodic phases with 662 and 665 being anodic phases).

Passive recharge alternating polarity stimulation 670 may include first pulse 672 and second pulse 674. In some examples, passive recharge alternating polarity stimulation 670 may include any number of pulses suitable to deliver a test stimulation to a patient. In some examples, active recharge alternating polarity stimulation 670 includes three, four, five, six, or any other suitable number of pulses to determine whether a signal of interest is present. First pulse 672 may include first cathodic phase 676 and first passive recharge phase 678 separated by an interphase interval. Processing circuitry 210 may control stimulation generation circuitry 202 to deliver first cathodic phase 676 as pulse of negative amplitude for a first duration, pause for a period of time shorter than the first duration, then processing circuitry 210 may control stimulation generation circuitry 202 to accept charge from the electrodes at first passive recharge phase 678 for a second duration suitable to reduce the built up charge from first cathodic phase 676. In some examples, the area under the curve, i.e., the amount of charge delivered or received, by first cathodic phase 676 and first passive recharge phase 678 may be equal. Processing circuitry 210 may control sensing circuitry 204 to record from electrode 116, 118 after controlling stimulation generation circuitry 202 to passively accept charge during first passive recharge phase 678. Processing circuitry 210 may wait for a period such as period 684. Period 684 may be the inverse of the frequency at which pulses are delivered. In some examples, the frequency may be 2-25 kHz or any other frequency suitable to deliver one or more test pulses.

After period 684, processing circuitry 210 may deliver second pulse 674. Second pulse 674 may include anodic phase 680 and second passive recharge phase 682 separated by an interphase interval. To deliver second pulse 674, processing circuitry 210 may control stimulation generation circuitry 202 to deliver anodic phase 680 as pulse of positive amplitude for a first duration, pause for a period of time shorter than the first duration, then processing circuitry 210 may control stimulation generation circuitry 202 to passively accept any built up charge for a second duration at second passive recharge phase 682 where the second duration is longer than the first duration and suitable to reduce the built up charge from anodic phase 680. In some examples, the area under the curve, i.e., the amount of charge delivered or received, by anodic phase 680 and second passive recharge phase 682 may be equal. In other examples, first pulse 672 and second pulse 674 may be flipped. In other examples, first pulse 672, or second pulse 674, may be repeated one or more additional times. In some examples, first pulse 672 and second pulse 674 may both be anodic-leading phase (i.e., both 676 and 680 are anodic phase) or may both be cathodic-leading phase (i.e., both 676 and 680 are cathodic pulses).

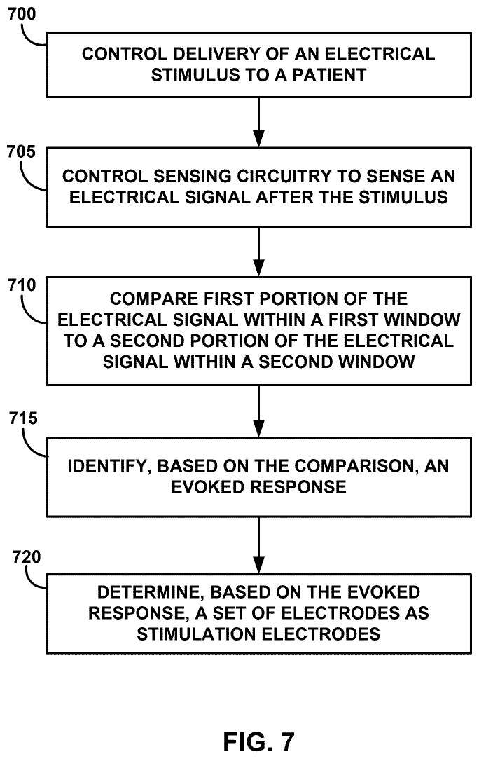

FIG. 7 is a flowchart illustrating an example operation of a device configured to select a stimulation electrode combination based on a comparison of sensed signals within different windows after a delivered stimulus. The example process of FIG. 7 will be described with respect to processing circuitry 210 of IMD 106, but other processing circuitry, such as processing circuitry 310 of external device 104 may additionally, or alternatively, perform at least some of the elements of the process.

As shown in the example of FIG. 7, processing circuitry 210 may control stimulation circuitry to deliver an electrical stimulus to a patient (700). Processing circuitry 210 may then control sensing circuitry of IMD 106 to sense an electrical signal after the electrical stimulus is delivered (705). Processing circuitry 210 may then compare a first portion of the electrical signal within a first sensing window to a second portion of the electrical signal within a second sensing window (710). The first sensing window may occur prior to the second sensing window, but the windows may be completely separate or partially overlapping in time.

Processing circuitry 210 may then identify, based on the comparison of the first portion to the second portion, an evoked response within the first sensing window that was elicited by the electrical stimulus (715). Processing circuitry 210 may then determine, based on the evoked response identified from the electrical signal, a set of electrodes of a plurality of electrodes as stimulation electrodes (720). Other stimulation parameters, such as amplitude, pulse width, pulse frequency, etc., may be also, or alternatively, determined based on the identification of the evoked signal. In some example, if the evoked signal was sensed, processing circuitry 210 may use the stimulation electrodes that delivered the stimulus as stimulation electrodes. In some examples, processing circuitry 210 may perform this process for multiple different electrode combinations delivering the stimulus. Processing circuitry 210 may use the largest evoked response and/or the evoked response detectable and the lowest amplitude of the stimulus suitable to achieve the response as the stimulation electrode combination. In some examples, processing circuitry 210 may use a similar comparison of these first and second windows (e.g., a sensing window and a baseline window) to identify when an evoked signal is present.

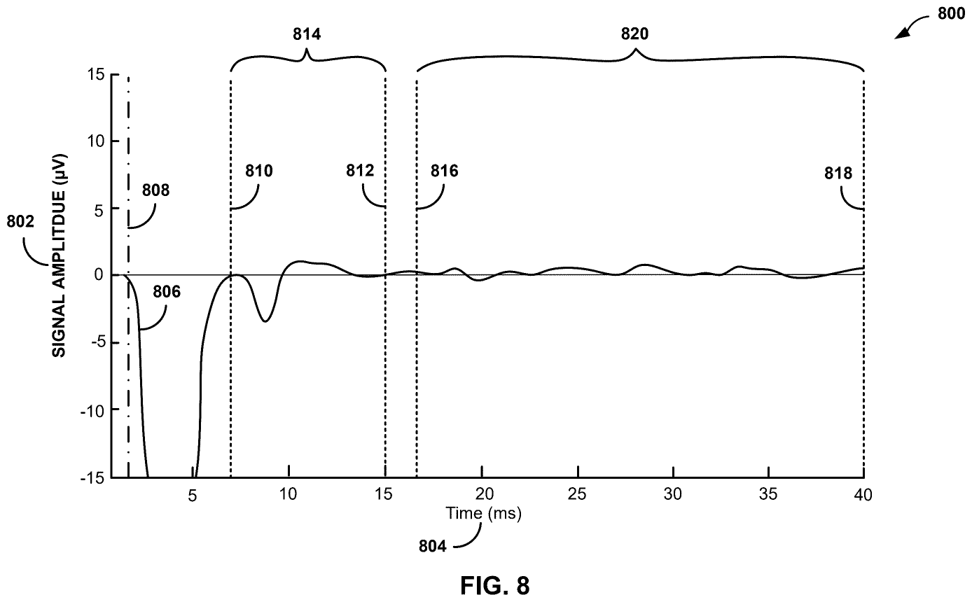

FIG. 8 is a graph of an example evoked response with an example first sensing window and an example second sensing window. The example evoked response of FIG. 8 will be described with respect to processing circuitry 210 of IMD 106, but other processing circuitry, such as processing circuitry 310 of external device 104 may additionally, or alternatively, be employed. Sensed data 800 illustrates a measurement (e.g., sensed evoked signals) obtained after an electrical stimulus has been delivered to a patient.

Sensed data 800 illustrates sensed electrical signal 806 after electrical stimulus 808 is delivered to target tissue of a patient. Electrical stimulus 808 may be an example the one or more stimulus pulses, of the stimulus of step 500 of FIG. 5, the first stimulus of step 600 of FIG. 6A, the second stimulus of step 610 of FIG. 6A, the stimulus of step 700 in FIG. 7, or any other stimulus. In some examples, such as the example described above in FIG. 1, the target tissue may include or be near spinal cord 128 and/or nerves 120 (e.g., pelvic nerves which may include a pudendal nerve or a sacral nerve), bulbospongiosus muscle, or any other nervous or muscle tissue that may be stimulated or from which physiological signals may be sensed of patient 112 through lead 114 (coupled to IMD 106 via connector 108). To record sensed data 800, processing circuitry 210 of IMD 106 may control sensing circuitry 204 to beginning measure a signal amplitude at electrodes 116, 118. Signal output measurements may be taken from one or more electrodes at a frequency of 2-25 kHz or any other frequency suitable to collect an electrical signal 806 response to electrical stimulus 808 which enables comparison between first window 814 and second window 820.

Signal amplitude 802 is measured in microvolts (μV). Signal amplitude 802 of electrical signal 806 may be an amalgamation of one or more measurements such as the one or more electrical signals measured as described above in step 405 of FIG. 4, steps 500-520 of FIG. 5, steps 600-625 of FIG. 6A, and or any other measurements of one or more signals. Time axis 804 represents a time axis over which signals are recorded. First window beginning 810 and first window end 812 bound first window 814. Specifically, first window beginning 810 defines a temporal beginning of first window 814 where first window end 812 defines a temporal end of first window 814. To calculate an average of the data of first window 814, processing circuitry 210 may copy electrical signal 806 to a working directory, blank data before first window beginning 810, blank data after first window end 812, and thereafter average electrical signal 806 of the working directory. Second window beginning 816 and first window end 818 bound second window 820. Specifically, second window beginning 816 defines a temporal beginning of second window 820 where second window end 818 defines a temporal end of second window 820. To calculate an average of the data of second window 820, processing circuitry 210 may copy electrical signal 806 to a working directory, blank data before second window beginning 816, blank data after second window end 818, and thereafter average electrical signal 806 of the working directory. The length of first window 814 may be pre-set, such as by a manufacturer or a clinician.

Processing circuitry 210 may additionally or alternatively select any length from a list of acceptable lengths for first window 814. In some examples, first window 814 may be 2 ms, 3 ms, 4 ms, 5 ms, 6 ms, 7 ms, 8 ms, 9 ms, 10 ms, 11 ms, 12 ms, or any other length suitable to record the evoked response. In some examples, first window beginning 810 may be a delay after electrical stimulus 808, such as 1 ms, 2 ms, 3 ms, 4 ms, 5 ms, 6 ms, 7 ms, 8 ms, or any other delay after processing circuitry 210 controls stimulation generation circuitry 202 to deliver one or more stimulation pulses which is suitable for detecting the stimulation response while excluding the stimulation pulse artifacts. In some examples, first window end 812 may occur within 10 ms, 11 ms, 12 ms, 13 ms, 14 ms, 15 ms, or any other suitable length of time after electrical stimulus 808 to record the evoked response.

In some examples, second window beginning 816 may occur after first window end 812. In some examples, second window beginning 816 may occur 0 ms, 1 ms, 2 ms, 3 ms, 4 ms, 5 ms, 6 ms, 7 ms, 8 ms, or any other suitable length of time after first window end 812. Processing circuitry 210 may control the timing of first window 814 and second window 820 such that a delay between the first sensing window 814 and the second sensing window 820 is within a period from 0 ms to 26 ms. In some examples, the delay between the first sensing window 814 and the second sensing window 820 is within a period from 0 ms to 8 ms. In some examples, second window beginning 816 may occur 15 ms, 16 ms, 17 ms, 18 ms, 19 ms, 20 ms, 21 ms, 22 ms, 23 ms, 24 ms, 25 ms, 26 ms, 27 ms, 28 ms, 29 ms, 30 ms, or any other suitable time after electrical stimulus 808. As such, the second sensing window 820 may have a second duration occurring within a period from 15 ms after an end of the electrical stimulus 808 to 30 ms after the end of the electrical stimulus 808. The length of second window 820 may be pre-set, such as by a manufacturer or a clinician. Processing circuitry 210 may additionally or alternatively select any length from a list of acceptable lengths for second window 820 such as 15 ms, 16 ms, 17 ms, 18 ms, 19 ms, 20 ms, 21 ms, 22 ms, 23 ms, 24 ms, 25 ms, 26 ms, 27 ms, 28 ms, 29 ms, 30 ms, or any other length suitable to record the baseline response.

In other examples, second window beginning 816 may occur before or concurrently with first window end 812 and as a result first window 814 overlaps in time with second window 820. In some examples, second window beginning 816 may occur 0 ms, 1 ms, 2 ms, 3 ms, 4 ms, 5 ms, 6 ms, 7 ms, 8 ms, or any other length of time up to the length of the first window before first window end 812.

The following examples are a non-limiting list of examples in accordance with one or more techniques of this disclosure.

Example 1. A system comprising: processing circuitry configured to: control stimulation circuitry to deliver an electrical stimulus to a patient; control sensing circuitry to sense an electrical signal after the electrical stimulus is delivered; compare a first portion of the electrical signal within a first sensing window to a second portion of the electrical signal within a second sensing window, wherein the first sensing window occurs prior to the second sensing window; identify, based on the comparison of the first portion to the second portion, an evoked response within the first sensing window that was elicited by the electrical stimulus; and determine, based on the evoked response identified from the electrical signal, a set of electrodes of a plurality of electrodes as stimulation electrodes.

Example 2. The system of Example 1, wherein the stimulation circuitry is configured to deliver the electrical stimulus to a target location comprising a sacral nerve of the patient.

Example 3. The system of any of Examples 1-2, wherein the plurality of electrodes are implanted in the patient.

Example 4. The system of any of Examples 1-3, wherein the processing circuitry is configured to control the stimulation circuitry to deliver the electrical stimulus by at least: controlling the stimulation circuitry to deliver a first pulse of the electrical stimulus via a first electrode of a first set of electrodes of the plurality of electrodes as a cathode and a second electrode of the first set of electrodes as an anode; and control the stimulation circuitry to deliver a second pulse of the electrical stimulus via the second electrode as the cathode and the first electrode as the anode, and wherein the second pulse comprises an active recharge pulse for the electrical stimulus.

Example 5. The system of any of Examples 1-4, wherein the electrical stimulus is a first electrical stimulus, the electrical signal is a first electrical signal, and the evoked response is a first evoked response, and wherein the processing circuitry is configured to: control the stimulation circuitry to deliver a second electrical stimulus to the patient, wherein a first polarity of the first electrical stimulus is opposite a second polarity of the second electrical stimulus; control the sensing circuitry to sense a second electrical signal after the second electrical stimulus is delivered; compare a first portion of the second electrical signal within a third sensing window to a second portion of the second electrical signal within a fourth sensing window, wherein the third sensing window occurs prior to the fourth sensing window; identify, based on the comparison of the first portion to the second portion of the second electrical signal, a second evoked response within the third sensing window that was elicited by the second electrical stimulus; and determine, based on the first evoked response and the second evoked response, the set of electrodes as stimulation electrodes.

Example 6. The system of any of Examples 1-5, wherein the sensing circuitry is further configured to apply at least one of an analog filter or a digital filter to the electrical signal.

Example 7. The system of any of Examples 1-6, wherein the processing circuitry is configured to: identify a plurality of evoked responses within sensing windows of respective electrical signals of a plurality of electrical signals, wherein the plurality of electrical signals comprises the electrical signal; and average the plurality of evoked responses to generate an average evoked response, wherein the processing circuitry is configured to determine the set of electrodes based on the average evoked response.

Example 8. The system of Example 7, wherein the processing circuitry is configured to generate the average evoked response by at least: comparing each evoked response within the first sensing window of each respective electrical signals to a threshold; and removing, based on the comparison, any evoked responses exceeding the threshold from the average evoked response.

Example 9. The system of any of Examples 1-8, wherein the first sensing window has a first duration occurring within 15 milliseconds after an end of the electrical stimulus and the second sensing window has a second duration occurring within a period from 15 ms after an end of the electrical stimulus to 30 ms after the end of the electrical stimulus.

Example 10. The system of Example 9, wherein a delay between the first sensing window and the second sensing window is within a period from 0 ms to 26 ms.

Example 11. The system of any of Examples 1-10, wherein the processing circuitry is configured to identify the evoked response within the first sensing window by at least: determining that a first feature of the first portion of the electrical signal within the first sensing window is greater than a second feature of the second portion of the electrical signal within the second sensing window; and responsive to the determination that the first feature is greater than the second feature, identifying the evoked response within the first sensing window.

Example 12. The system of any of Examples 1-11, wherein the processing circuitry is configured to compare the first portion to the second portion by at least comparing a first peak-to-peak amplitude of the first portion to a second peak-to-peak amplitude of the second portion.

Example 13. The system of any of Examples 1-12, further comprising an implantable medical device comprising the stimulation circuitry and the sensing circuitry, and wherein the stimulation circuitry is configured to deliver electrical stimulation therapy via the stimulation electrodes.

Example 14. A method comprising: controlling, by processing circuitry, stimulation circuitry to deliver an electrical stimulus to a patient; controlling, by the processing circuitry, sensing circuitry to sense an electrical signal after the electrical stimulus is delivered; comparing, by the processing circuitry, a first portion of the electrical signal within a first sensing window to a second portion of the electrical signal within a second sensing window, wherein the first sensing window occurs prior to the second sensing window; identifying, by the processing circuitry and based on the comparison of the first portion to the second portion, an evoked response within the first sensing window that was elicited by the electrical stimulus; and determining, by the processing circuitry and based on the evoked response identified from the electrical signal, a set of electrodes of a plurality of electrodes as stimulation electrodes.

Example 15. The method of Example 14, wherein the stimulation circuitry is configured to deliver the electrical stimulus to a target location comprising a sacral nerve of the patient, and wherein the plurality of electrodes are implanted within the patient.

Example 16. The method of any of Examples 14-15, wherein the processing circuitry is configured to control the stimulation circuitry to deliver the electrical stimulus by at least: controlling the stimulation circuitry to deliver a first pulse of the electrical stimulus via a first electrode of a first set of electrodes of the plurality of electrodes as a cathode and a second electrode of the first set of electrodes as an anode; and control the stimulation circuitry to deliver a second pulse of the electrical stimulus via the second electrode as the cathode and the first electrode as the anode, and wherein the second pulse comprises an active recharge pulse for the electrical stimulus.

Example 17. The method of any of Examples 14-16, wherein the electrical stimulus is a first electrical stimulus, the electrical signal is a first electrical signal, and the evoked response is a first evoked response, and wherein the method further comprises: controlling the stimulation circuitry to deliver a second electrical stimulus to the patient, wherein a first polarity of the first electrical stimulus is opposite a second polarity of the second electrical stimulus; controlling the sensing circuitry to sense a second electrical signal after the second electrical stimulus is delivered; comparing a first portion of the second electrical signal within a third sensing window to a second portion of the second electrical signal within a fourth sensing window, wherein the third sensing window occurs prior to the fourth sensing window; identifying, based on the comparison of the first portion to the second portion of the second electrical signal, a second evoked response within the third sensing window that was elicited by the second electrical stimulus; and determining, based on the first evoked response and the second evoked response, the set of electrodes of the plurality of electrodes as stimulation electrodes.

Example 18. The method of any of Examples 14-17, wherein the processing circuitry is configured to: identify a plurality of evoked responses within first sensing windows of respective electrical signals of a plurality of electrical signals, wherein the plurality of electrical signals comprises the electrical signal; and average the plurality of evoked responses to generate an average evoked response, wherein the processing circuitry is configured to determine the set of electrodes based on the average evoked response.

Example 19. The method of any of Examples 14-18, wherein the first sensing window has a first duration occurring within 15 milliseconds after an end of the electrical stimulus and the second sensing window has a second duration occurring within a period from 15 ms after an end of the electrical stimulus to 30 ms after the end of the electrical stimulus, wherein a delay between the first sensing window and the second sensing window is within a period from 0 ms to 26 ms.

Example 20. A non-transitory computer-readable storage medium comprising instructions that, when executed, causes processing circuitry to: control stimulation circuitry to deliver an electrical stimulus to a patient; control sensing circuitry to sense an electrical signal after the electrical stimulus is delivered; compare a first portion of the electrical signal within a first sensing window to a second portion of the electrical signal within a second sensing window, wherein the first sensing window occurs prior to the second sensing window; identify, based on the comparison of the first portion to the second portion, an evoked response within the first sensing window that was elicited by the electrical stimulus; and determine, based on the evoked response identified from the electrical signal, a set of electrodes of a plurality of electrodes as stimulation electrodes.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A system comprising:
processing circuitry configured to:
control stimulation circuitry to deliver an electrical stimulus to a patient;
control sensing circuitry to sense an electrical signal after the electrical stimulus is delivered;
compare a first portion of the electrical signal within a first sensing window to a second portion of the electrical signal within a second sensing window, wherein the first sensing window occurs prior to the second sensing window;
identify, based on the comparison of the first portion to the second portion, an evoked response within the first sensing window that was elicited by the electrical stimulus; and
determine, based on the evoked response identified from the electrical signal, a set of electrodes of a plurality of electrodes as stimulation electrodes.

2. The system of claim 1, wherein the stimulation circuitry is configured to deliver the electrical stimulus to a target location comprising a sacral nerve of the patient.

3. The system of claim 1, wherein the plurality of electrodes are implanted in the patient.

4. The system of claim 1, wherein the processing circuitry is configured to control the stimulation circuitry to deliver the electrical stimulus by at least:
controlling the stimulation circuitry to deliver a first pulse of the electrical stimulus via a first electrode of a first set of electrodes of the plurality of electrodes as a cathode and a second electrode of the first set of electrodes as an anode; and
control the stimulation circuitry to deliver a second pulse of the electrical stimulus via the second electrode as the cathode and the first electrode as the anode, and wherein the second pulse comprises an active recharge pulse for the electrical stimulus.

5. The system of claim 1, wherein the electrical stimulus is a first electrical stimulus, the electrical signal is a first electrical signal, and the evoked response is a first evoked response, and wherein the processing circuitry is configured to:
control the stimulation circuitry to deliver a second electrical stimulus to the patient, wherein a first polarity of the first electrical stimulus is opposite a second polarity of the second electrical stimulus;
control the sensing circuitry to sense a second electrical signal after the second electrical stimulus is delivered;
compare a first portion of the second electrical signal within a third sensing window to a second portion of the second electrical signal within a fourth sensing window, wherein the third sensing window occurs prior to the fourth sensing window;
identify, based on the comparison of the first portion to the second portion of the second electrical signal, a second evoked response within the third sensing window that was elicited by the second electrical stimulus; and
determine, based on the first evoked response and the second evoked response, the set of electrodes as stimulation electrodes.

6. The system of claim 1, wherein the sensing circuitry is further configured to apply at least one of an analog filter or a digital filter to the electrical signal.

7. The system of claim 1, wherein the processing circuitry is configured to: identify a plurality of evoked responses within sensing windows of respective electrical signals of a plurality of electrical signals, wherein the plurality of electrical signals comprises the electrical signal; and
average the plurality of evoked responses to generate an average evoked response, wherein the processing circuitry is configured to determine the set of electrodes based on the average evoked response.

8. The system of claim 7, wherein the processing circuitry is configured to generate the average evoked response by at least:
comparing each evoked response within the first sensing window of each respective electrical signals to a threshold; and
removing, based on the comparison, any evoked responses exceeding the threshold from the average evoked response.

9. The system of claim 1, wherein the first sensing window has a first duration occurring within 15 milliseconds after an end of the electrical stimulus and the second sensing window has a second duration occurring within a period from 15 ms after an end of the electrical stimulus to 30 ms after the end of the electrical stimulus.

10. The system of claim 9, wherein a delay between the first sensing window and the second sensing window is within a period from 0 ms to 26 ms.

11. The system of claim 1, wherein the processing circuitry is configured to identify the evoked response within the first sensing window by at least:
determining that a first feature of the first portion of the electrical signal within the first sensing window is greater than a second feature of the second portion of the electrical signal within the second sensing window; and
responsive to the determination that the first feature is greater than the second feature, identifying the evoked response within the first sensing window.

12. The system of claim 1, wherein the processing circuitry is configured to compare the first portion to the second portion by at least comparing a first peak-to-peak amplitude of the first portion to a second peak-to-peak amplitude of the second portion.

13. The system of claim 1, further comprising an implantable medical device comprising the stimulation circuitry and the sensing circuitry, and wherein the stimulation circuitry is configured to deliver electrical stimulation therapy via the stimulation electrodes.

14. A method comprising:
controlling, by processing circuitry, stimulation circuitry to deliver an electrical stimulus to a patient;
controlling, by the processing circuitry, sensing circuitry to sense an electrical signal after the electrical stimulus is delivered;
comparing, by the processing circuitry, a first portion of the electrical signal within a first sensing window to a second portion of the electrical signal within a second sensing window, wherein the first sensing window occurs prior to the second sensing window;
identifying, by the processing circuitry and based on the comparison of the first portion to the second portion, an evoked response within the first sensing window that was elicited by the electrical stimulus; and
determining, by the processing circuitry and based on the evoked response identified from the electrical signal, a set of electrodes of a plurality of electrodes as stimulation electrodes.

15. The method of claim 14, wherein the stimulation circuitry is configured to deliver the electrical stimulus to a target location comprising a sacral nerve of the patient, and wherein the plurality of electrodes are implanted within the patient.

16. The method of claim 14, wherein the processing circuitry is configured to control the stimulation circuitry to deliver the electrical stimulus by at least:
controlling the stimulation circuitry to deliver a first pulse of the electrical stimulus via a first electrode of a first set of electrodes of the plurality of electrodes as a cathode and a second electrode of the first set of electrodes as an anode; and
control the stimulation circuitry to deliver a second pulse of the electrical stimulus via the second electrode as the cathode and the first electrode as the anode, and wherein the second pulse comprises an active recharge pulse for the electrical stimulus.

17. The method of claim 14, wherein the electrical stimulus is a first electrical stimulus, the electrical signal is a first electrical signal, and the evoked response is a first evoked response, and wherein the method further comprises:
controlling the stimulation circuitry to deliver a second electrical stimulus to the patient, wherein a first polarity of the first electrical stimulus is opposite a second polarity of the second electrical stimulus;
controlling the sensing circuitry to sense a second electrical signal after the second electrical stimulus is delivered;
comparing a first portion of the second electrical signal within a third sensing window to a second portion of the second electrical signal within a fourth sensing window, wherein the third sensing window occurs prior to the fourth sensing window;
identifying, based on the comparison of the first portion to the second portion of the second electrical signal, a second evoked response within the third sensing window that was elicited by the second electrical stimulus; and
determining, based on the first evoked response and the second evoked response, the set of electrodes of the plurality of electrodes as stimulation electrodes.

18. The method of claim 14, wherein the processing circuitry is configured to:
identify a plurality of evoked responses within first sensing windows of respective electrical signals of a plurality of electrical signals, wherein the plurality of electrical signals comprises the electrical signal; and
average the plurality of evoked responses to generate an average evoked response, wherein the processing circuitry is configured to determine the set of electrodes based on the average evoked response.

19. The method of claim 14, wherein the first sensing window has a first duration occurring within 15 milliseconds after an end of the electrical stimulus and the second sensing window has a second duration occurring within a period from 15 ms after an end of the electrical stimulus to 30 ms after the end of the electrical stimulus, wherein a delay between the first sensing window and the second sensing window is within a period from 0 ms to 26 ms.

20. A non-transitory computer-readable storage medium comprising instructions that, when executed, causes processing circuitry to:
control stimulation circuitry to deliver an electrical stimulus to a patient;
control sensing circuitry to sense an electrical signal after the electrical stimulus is delivered;
compare a first portion of the electrical signal within a first sensing window to a second portion of the electrical signal within a second sensing window, wherein the first sensing window occurs prior to the second sensing window;
identify, based on the comparison of the first portion to the second portion, an evoked response within the first sensing window that was elicited by the electrical stimulus; and
determine, based on the evoked response identified from the electrical signal, a set of electrodes of a plurality of electrodes as stimulation electrodes.
